(19) United States
(12) Patent Application Publication
(10) Pub. No.: US 2020/0179567 A1
(43) Pub. Date: Jun. 11, 2020

(54) REGIONALLY SPECIFIC TISSUE-DERIVED EXTRACELLULAR MATRIX

(71) Applicant: The Trustees of Columbia University in the City of New York, New York, NY (US)

(72) Inventors: John O'Neill, New York, NY (US); Gordana Vunjak-Novakovic, New York, NY (US)

(21) Appl. No.: 16/054,162

(22) Filed: Aug. 3, 2018

Related U.S. Application Data
(63) Continuation of application No. PCT/US2017/016596, filed on Feb. 3, 2017.
(60) Provisional application No. 62/291,926, filed on Feb. 5, 2016.

(51) Int. Cl. A61L 27/36; A61L 27/54; A61L 27/52; A61L 27/56; A61L 27/38

(52) U.S. Cl. CPC A61L 27/3633; A61L 27/54; A61L 27/3834; A61L 27/56; A61L 27/52

(57) ABSTRACT

Region-specific extracellular matrix (ECM) biomaterials are provided. Such materials include acellular scaffolds, sponges, solutions, hydrogels, fibers and bio-inks suitable for cell culture.

### CROSS REFERENCE TO RELATED APPLICATIONS

This application is a continuation of International Application No. PCT/US2017/016596, filed on Feb. 3, 2017, which claims priority to U.S. Provisional Patent Application Ser. No. 62/291,926, filed Feb. 5, 2016, the content of which is hereby incorporated by reference in its entirety.

### FIELD OF THE DISCLOSED SUBJECT MATTER

The disclosed subject matter relates to biomaterials derived from healthy, diseased, or transgenic region-specific tissue extracellular matrix. Particularly, the presently disclosed subject matter relates to methods to isolate, decellularize, and process regions or anatomical features of various organs from various sources (including human and animal; fetal, juvenile, and adult; healthy, diseased, and transgenic) into various formats including: acellular scaffolds, sponges, hydrogels, liquid solutions, fibers (e.g., electrospun fibers), and bio-ink (e.g., printable bio-ink). The presently disclosed subject matter further relates to such scaffolds, sponges, hydrogels, solutions, fibers, and bio-ink suitable for cell culture.

### BACKGROUND

Extracellular matrix (ECM) provides cells with a scaffold with tissue-specific cues (e.g.,molecular, structural, biomechanical) that mediate cell function. Cells reside in specialized ECM niches, such as stem cells in the papilla region of the kidney, in the epithelium of the lung and in the embryonic heart. Currently, it is not possible to re-create the complex environment of tissues or organs using synthetic materials.

Accordingly, there remains a need for a medium that provides an environment suitable for the growth of cells for a variety of different tissues, such as the adrenal gland, amnion, bladder, blood vessel, bone, brain, breast, cartilage, chorion, connective tissue, esophagus, eye, fat, heart, kidney, large intestine, larynx, ligament, liver, lung, lymph node, microvasculature, muscle, omentum, ovary, pancreas, placenta membrane, prostate, skin, small intestine, smooth muscle, spinal cord, spleen, stomach, tendon, testis, thymus, umbilical cord, uterus, or Wharton's Jelly.

### SUMMARY

Native extracellular matrix (ECM) that is formed and maintained by resident cells is of great interest for cell culture and cell delivery. As set forth below, specialized bioengineered niches for cells can be established using regionally specific ECM-derived material. Although various embodiments refer to one tissue as an example, the methods and products set forth herein are applicable to various tissues. In one aspect, a specific region is isolated and processed to remove cellular material while preserving the composition, architecture, and mechanical properties of the tissue matrix. This decellularized matrix may be sectioned to produce the first format—an acellular scaffold. Additional processing of the decellularized matrix results in the solution format. The solution can then be further processed into one of a hydrogel, a porous sponge, a surface coating, a media supplement or a bio-ink.

As an exemplary system, kidney is selected because of the high regional diversification of its tissue matrix. By preparing the ECM from three specialized regions of the kidney (e.g., cortex, medulla and papilla; the whole kidney, heart and bladder as controls) in three formats: (i) acellular ECM scaffolds (intact sheets of decellularized ECM), (ii) ECM hydrogels, and (iii) ECM solutions (solubilized ECM), it is shown how the structure and composition of ECM affect the function of kidney stem cells (with mesenchymal stem cells, or MSCs, serving as control). All three forms of the ECM regulate kidney stem cell (KSC) function, with differential structural and compositional effects. KSCs cultured on papilla ECM consistently display higher metabolic activity and differences in cell morphology, alignment, proliferation and structure formation as compared to cortex and medulla ECM, the effects not observed in corresponding MSC cultures. Thus, tissue- and region-specific ECM can provide an effective substrate for in vitro cell culture studies.

As another exemplary system, lung is selected because of the marked regional differences of its tissue composition and matrix. Three different methods of decellularization and the cultivation of three different types of human cells (lung fibroblasts, small airway epithelial cells, mesenchymal cells) were evaluated. By preparing the ECM from three specialized regions of the lung (e.g. airway cartilage, airway mucosa, and parenchyma) in two formats (i) acellular ECM scaffolds and (ii) ECM hydrogels, it is shown how the structure and composition of ECM affect the function of lung or mesenchymal stem cells. Acellular ECM scaffolds and ECM hydrogels regulate the functions of the lung and mesenchymal stem cells, through their differential structural, mechanical, and compositional effects. Lung stem cells cultured on parenchyma lung ECM display growth, differentiation, and functional uptake of surfactant. Mesenchymal stem cells cultured on airway cartilage matrix display higher chondrogenesis, matrix deposition, and expression of collagen II. Thus tissue and region specific ECM can provide an effective substrate for in vitro cell culture studies.

Another exemplary system comprises cardiac extracellular matrices. Native heart extracellular matrix (ECM) can direct cardiac differentiation of human embryonic stem cells (hESCs) in vitro without the addition of soluble factors. A series of hydrogels was prepared from decellularized ECM from porcine hearts by mixing ECM and collagen type I at varying ratios. hESC-derived repair cells can potentially be implanted into the damaged area of the heart to promote neovascularization and myogenesis. Described herein are three-dimensional scaffolds that mimic the native ECM, to guide and promote myocardial regeneration. By providing the necessary microenvironment, ECM regulates many cellular activities, including migration and differentiation. One way to provide the necessary biochemical cues for cardiac regeneration is to use hydrogels derived from native cardiac tissue matrix mixed with collagen.

Similarly, tissues from other organs of various sources are processed into various formats. Specific regions or anatomical features of exemplary organs including the adrenal gland, amnion, bladder, blood vessel, bone, brain, breast, cartilage, chorion, connective tissue, esophagus, eye, fat, heart, kidney, large intestine, larynx, ligament, liver, lung, lymph node, microvasculature, muscle, omentum, ovary, pancreas, placenta membrane, prostate, skin, small intestine, smooth muscle, spinal cord, spleen, stomach, tendon, testis, thymus, umbilical cord, uterus, or Wharton's Jelly can be processed into various formats. These tissues are drawn from various sources including human and animal; fetal, juvenile, and adult; and healthy, diseased, and transgenic tissues. As discussed herein, these materials can be processed into formats including acellular scaffolds, sponges, hydrogels, fibers (i.e obtained by electrospinning), bio-inks (e.g., obtained by printing) and other solution preparations.

The resulting biomaterials comprise an array of highly specific materials composed of native organ region extracellular matrix. They can be used to grow, maintain, or differentiate organ- or region-specific cells in culture or to stimulate repair or regeneration in vivo. Specific applications of interest include, but are not limited to:

In vitro three-dimensional culture/testing of cells in acellular scaffolds, in sponges, in hydrogels, in fibers (e.g., electrospun fibers);

In vitro culture/testing of cells with culture medium supplemented with matrix solution;

In vitro surface coating (adsorption) of matrix solution of cell culture flask/dish by matrix surface coating to increase cell attachment and growth;

In vitro guided differentiation of embryonic stem cells or induced pluripotent stem cells into organ-specific cells;

In vivo injection/delivery of therapeutic cells, drugs, other soluble factors via hydrogel;

In vivo implantation of acellular scaffolds with or without cells for tissue/organ regeneration studies; and/or

3D printing of tissue/organ constructs using tissue-specific matrix bio-ink.

Exemplary compositions include, without limitation, acellular matrix scaffolds of any desired size and shape; sponge matrix products of any desired size and shape; cell culture wells/plates coated with matrix surface coating; liquid matrix solutions; a kit containing matrix, reagents, and instructions for reconstituting hydrogel or media supplements; and/or customized matrix biomaterials (format, size, properties, etc.).

To achieve these and other advantages and in accordance with the purpose of the disclosed subject matter, as embodied and broadly described, the disclosed subject matter includes a culture medium. In some embodiments, the culture medium includes a scaffold and the scaffold comprises a decellularized extracellular matrix. In some embodiments, the scaffold comprises a planar sheet. In some embodiments, the decellularized extracellular matrix is a region-specific extracellular matrix. In some embodiments, the decellularized extracellular matrix is an organ-specific extracellular matrix. In some embodiments, the decellularized extracellular matrix is extracellular matrix of a region of an organ. In some embodiments, the decellularized extracellular matrix is selected from the group consisting of: adrenal gland extracellular matrix, amnion extracellular matrix, bladder extracellular matrix, blood vessel extracellular matrix, brain extracellular matrix, breast extracellular matrix, bone extracellular matrix, esophagus extracellular matrix, cartilage extracellular matrix, chorion extracellular matrix, connective tissue extracellular matrix, eye extracellular matrix, fat extracellular matrix, heart extracellular matrix, kidney extracellular matrix, large intestine extracellular matrix, larynx extracellular matrix, ligament extracellular matrix, liver extracellular matrix, lung extracellular matrix, lymph node extracellular matrix, microvasculature extracellular matrix, muscle extracellular matrix, omentum extracellular matrix, ovary extracellular matrix, parathyroid extracellular matrix, pancreas extracellular matrix, placenta extracellular matrix, prostate extracellular matrix, skin extracellular matrix, small intestine extracellular matrix, smooth muscle extracellular matrix, spinal cord extracellular matrix, spleen extracellular matrix, stomach extracellular matrix, tendon extracellular matrix, testes extracellular matrix, thymus extracellular matrix, thyroid extracellular matrix, umbilical cord extracellular matrix, uterus extracellular matrix and Wharton's Jelly extracellular matrix.

In another aspect of the present subject matter, a kit for supplementing culture medium is provided. The kit includes a solution and at least one reagent. The solution comprises decellularized extracellular matrix. At least one reagent is adapted to reconstitute the solution into a hydrogel. In some embodiments, the reagent comprises phosphate-buffered saline or sodium hydroxide. In some embodiments, the decellularized extracellular matrix is a region-specific extracellular matrix. In some embodiments, the decellularized extracellular matrix is an organ-specific extracellular matrix. In some embodiments, the decellularized extracellular matrix is extracellular matrix of a region of an organ. In some embodiments, the decellularized extracellular matrix is selected from the group consisting of: adrenal gland extracellular matrix, amnion extracellular matrix, bladder extracellular matrix, blood vessel extracellular matrix, brain extracellular matrix, breast extracellular matrix, bone extracellular matrix, esophagus extracellular matrix, cartilage extracellular matrix, chorion extracellular matrix, connective tissue extracellular matrix, eye extracellular matrix, fat extracellular matrix, heart extracellular matrix, kidney extracellular matrix, large intestine extracellular matrix, larynx extracellular matrix, ligament extracellular matrix, liver extracellular matrix, lung extracellular matrix, lymph node extracellular matrix, microvasculature extracellular matrix, muscle extracellular matrix, omentum extracellular matrix, ovary extracellular matrix, parathyroid extracellular matrix, pancreas extracellular matrix, placenta extracellular matrix, prostate extracellular matrix, skin extracellular matrix, small intestine extracellular matrix, smooth muscle extracellular matrix, spinal cord extracellular matrix, spleen extracellular matrix, stomach extracellular matrix, tendon extracellular matrix, testes extracellular matrix, thymus extracellular matrix, thyroid extracellular matrix, umbilical cord extracellular matrix, uterus extracellular matrix and Wharton's Jelly extracellular matrix.

In other aspects of the present subject matter, culture media are provided. In some embodiments, the culture media include a hydrogel, the hydrogel comprising decellularized extracellular matrix. In some embodiments, the culture media include solubilized decellularized extracellular matrix. In some embodiments, the culture media include a sponge, the sponge comprising decellularized extracellular matrix. In some embodiments, the culture media include fibers (e.g., electrospun fibers), the fibers comprising decellularized extracellular matrix. In some embodiments, the culture media include bio-ink (e.g., printable bio-ink), the bio-ink comprising decellularized extracellular matrix.

In another aspect, the disclosed subject matter includes a method of creating a hydrogel. A portion of an organ is extracted. The organ portion is decellularized to yield extracellular matrix. The extracellular matrix is powdered to yield a powder. The powder is digested to yield a digest. The digest is reconstituted into a hydrogel.

In yet another aspect, the described subject matter includes methods of using the regionally specific tissue derived ECM for clinical applications. It can provide an extracellular environment for cells and serve as a substitute for damaged tissue. For example, in cardiovascular tissue engineering, the ECM can be in the form of a sponge, hydrogel, fiber, patch, or an interposition graft to promote tissue reconstruction. In some applications, a sustained release of basic fibroblast growth factor-2 (FGF-2) may be incorporated in the material for delivery of therapeutic agents from the biomaterial. Potential clinical advantages for using the regionally specific ECM include, but are not limited to, promotion of myocardial remodeling, promotion of articular cartilage repair, promotion of cortical bone repair, promotion of skin wound repair and remodeling, promotion of smooth muscle and mucosal growth and regeneration of fistula (e.g., tracheoesophageal fistula), promotion of the growth of fat tissue reconstruction, promotion of endovascular repair and recovery; delivery of therapeutic cells and agents into the joints to treat pain and arthritis, into the pulmonary airways to treat acute and chronic lung injury, into the eye to treat corneal injury, into the kidney to treat acute kidney injury, into the skin to reduce scaring and the effects of aging.

The purpose and advantages of the disclosed subject matter will be set forth in and apparent from the description herein, as well as will be learned by practice of the disclosed subject matter. Additional advantages of the disclosed subject matter will be realized and attained by the methods and systems particularly pointed out in the written description and claims hereof, as well as from the appended drawings.

It is to be understood that both the foregoing general description and the following detailed description are exemplary and are intended to provide further explanation of the disclosed subject matter claimed.

### BRIEF DESCRIPTION OF THE DRAWINGS

A detailed description of various aspects, features, and embodiments of the subject matter described herein is provided with reference to the accompanying drawings, which are briefly described below. The drawings are illustrative and are not necessarily drawn to scale, with some components and features being exaggerated for clarity. The drawings illustrate various aspects and features of the present subject matter and may illustrate one or more embodiment(s) or example(s) of the present subject matter in whole or in part.

FIGS. 1A-C illustrate the stem cell niche of the kidney.

FIGS. 2A-F illustrate removal of cellular material and preservation of ECM in decellularized kidney regions.

FIGS. 3A-B illustrate ultrastructure of native and decellularized kidney regions.

FIGS. 4A-B illustrate collagen IV and fibronectin native and decellularized kidney regions.

FIGS. 5A-F illustrate DNA and metabolic activity of KSCs and MSCs in the presence of solubilized regionally specific kidney ECM.

FIGS. 6A-H illustrate metabolic activity, DNA content, and rhodamine-phalloidin/DAPI staining of KSCs and MSCs on regional kidney ECM hydrogels.

FIGS. 7A-H illustrates metabolic activity, DNA content, and rhodamine-phalloidin/DAPI staining of KSCs and MSCs on region-specific decellularized kidney ECM sheets.

FIG. 8 illustrates live/dead and rhodamine/phalloidin staining of KSCs and MSCs on regionally specific decellularized kidney region ECM sheets.

FIGS. 9A-D illustrate region-specific effects of ECM on metabolism of kidney stem cells.

FIGS. 10A-C illustrate characterization of solubilized kidney region ECM and ECM hydrogels.

FIGS. 11A-B illustrate chemotaxis of KSCs in the presence of solubilized kidney region ECM.

FIGS. 12A-C show characterization of lung decellularization using three different methods.

FIGS. 13A-C show characterization of collagen, sulfated glycosaminoglycans, and elastin in lung ECMs.

FIG. 14 shows histologic evaluation of human and porcine lung tissues after decellularization.

FIGS. 15A-D show distributions of extracellular matrix proteins of native tissue compared to ECMs.

FIG. 16 shows scanning electron micrographs of the ultrastructural morphologies of human and porcine LECM before and after decellularization.

FIG. 17 illustrates schematically preparation of decellularized human and porcine LECMs for mechanical characterization.

FIGS. 18A-D show mechanical properties of lung tissue.

FIGS. 19A-C shows growth curves of three human cell types on lung scaffolds decellularized by CHAPS.

FIGS. 20A-D illustrates viability and metabolic activity of three human cell types on lung scaffolds decellularized by CHAPS.

FIG. 21 shows a protocol for culturing cells to examine the effect of adding dexamethasone, 8-bromo-cAMP and isobutylmethylxanthine (DCI).

FIG. 22 shows micrographic images of the generation of airway and lung epitheal cells from human pluripotent stem cells after being seeded on decellularized human lung matrix.

FIG. 23 shows a model system for cultivation of human embryonic derived cardiomyocytes encapsulated in hydrogels.

FIG. 24 shows schematically the derivation of cardiac ECM digests.

FIGS. 25A-E show gelation and mechanical properties of cardiac hydrogels.

FIGS. 26A-B show the characterization of EBs.

FIGS. 27A-B show levels of expression of cardiac troponin T (cTnT).

FIGS. 28A-B show characterization of contractile behavior.

FIG. 29 shows confocal images of cardiac markers in human ESC derived cardiomyocytes cultured in different hydrogels.

FIG. 30 shows tissue-specific biomaterials and several products that can be formed therefrom.

### DETAILED DESCRIPTION OF EXEMPLARY EMBODIMENTS

The present disclosure provides ECM biomaterials in various formats including acellular scaffolds, sponges, hydrogels, solutions, fibers (e.g., electrospun fibers), or bio-inks (e.g., printable bio-inks). These materials are derived from various tissues such as adrenal gland, amnion, bladder, blood vessel, bone, brain, breast, cartilage, chorion, connective tissue, esophagus, eye, fat, heart, kidney, large intestine, larynx, ligament, liver, lung, lymph node, microvasculature, muscle, omentum, ovary, pancreas, placenta membrane, prostate, skin, small intestine, smooth muscle, spinal cord, spleen, stomach, tendon, testis, thymus, umbilical cord, uterus, or Wharton's Jelly. Tissues may be from various sources such as human and animal; fetal, juvenile, and adult; healthy, diseased, and transgenic.

These ECM biomaterials modulate stem cells in a region-specific manner. For example, data show that there is a significant degree of recognition and specificity between adult kidney stem cells and their extracellular environment. KSCs showed significantly higher proliferation and higher metabolic activity in kidney ECM when compared to KSCs in ECM from other organs. In addition, KSCs showed lower proliferation and higher metabolic activity when cultured in papilla ECM (kidney stem cell niche) compared to medulla and cortex ECM. The decrease in cell proliferation by ECM is of great interest since in vivo the kidney papilla shows little cycling activity. These effects were not observed with bone marrow-derived MSCs cultured under the same conditions, and the observed differences were independent of the form of the EMC, i.e., sheet vs. hydrogel vs. solubilized form.

Reference will now be made in detail to exemplary embodiments of the disclosed subject matter. Methods and corresponding steps of the disclosed subject matter will be described in conjunction with the detailed description of the system.

The extracellular matrix (ECM), the native scaffolding material secreted and maintained by residents cells, provides an ideal microenvironment for the cells with tissue-specific physical and molecular cues mediating cell proliferation, differentiation, gene expression, migration, orientation, and assembly. Functional and structural components within the ECM contribute to the extracellular environment specific to each tissue and organ. The complexity of the ECM has proven difficult to recapitulate in its entirety. Mimicking just the ECM structure using synthetic biomaterials or mimicking composition by adding purified ECM components is possible. While offering structural mimics, synthetic biomaterials can alter cell behavior (i.e. proliferation, differentiation, gene expression, migration, orientation, and assembly) in culture in vitro and potentially generate cytotoxic by-products at the site of implantation, leading to poor wound healing or an inflammatory environment.

An alternative to synthetic biomaterials is to directly isolate the native ECM from the tissue of interest via the removal of cells and cellular remnants. ECM scaffolds may be derived from a variety of tissues such as adrenal gland, amnion, bladder, blood vessel, bone, brain, breast, cartilage, chorion, connective tissue, esophagus, eye, fat, heart, kidney, large intestine, larynx, ligament, liver, lung, lymph node, microvasculature, muscle, omentum, ovary, pancreas, placenta membrane, prostate, skin, small intestine, smooth muscle, spinal cord, spleen, stomach, tendon, testis, thymus, umbilical cord, uterus, or Wharton's Jelly. ECM-derived biomaterials can be processed into scaffolds (such as acellular scaffolds or sponges) with appropriate compositions and structures for cell culture and tissue engineering. Furthermore, ECM scaffolds gradually degrade while promoting tissue remodeling at the site of implantation. Due to their biocompatibility and their ability to modulate the host tissue and immune response, ECM scaffolds are suitable for tissue engineering and regenerative medicine applications. ECM scaffolds can be derived from various sources such as human and animal; fetal, juvenile, and adult; healthy, diseased, and transgenic tissues.

ECM-based scaffolds can also be used to regulate the differentiation and maintenance of stem cells and their differentiated progeny. Stem cells normally reside within unique and highly regulated ECM serving as a niche. Complex tissues such as heart, lung, and kidney may be subjected to decellularization to obtain native-ECM scaffolds without particular regard for any specific region of the organ or preservation of potential stem cell niches. However, cells native to a particular region of the organ (e.g., heart: atrial myocardium, ventricular myocardium, cardiovascular endothelium; lung: airway cartilage, large pulmonary airway mucosa, small pulmonary airway mucosa, parenchymal alveoli; kidney: renal cortex, medulla, papilla) display ECM recognition and specificity. Extending this site-specific recognition to stem cells renders the choice of matrix an important consideration.

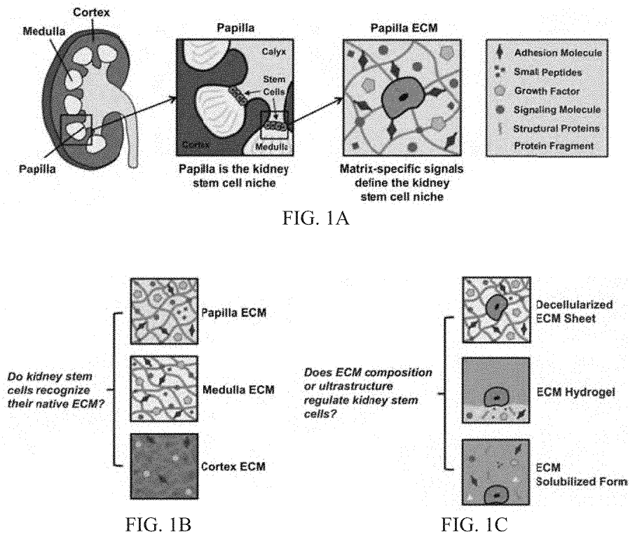

Referring to FIGS. 1A-1C, the stem cell niche of the kidney is illustrated. In FIG. 1A, the renal papilla is the stem cell niche in the kidney, defined by region-specific cues in the extracellular matrix of the papilla. In FIG. 1B, the renal cortex, medulla, and papilla were dissected and decellularized separately to obtain region-specific kidney ECM. In FIG. 1C, ECM was prepared in sheets, hydrogels, and solubilized form for cultivation of the two types of stem cells (kidney stem cells and mesenchymal stem cells). To obtain regionally specific kidney ECM sheets, kidney regions can be dissected before decellularization or the regional matrix is punched from whole decellularized kidney sections. Alternatively, pre-sectioned regions were decellularized, snap-frozen in liquid nitrogen, ground into a fine powder, lyophilized, and pepsin-digested to yield regionally specific kidney ECM digests that were neutralized to obtain solubilized ECM forms. By treating these digests with salt, base, and heat, we obtained regionally specific kidney ECM hydrogels. Kidney stem cells (KSCs) that are native to the papilla were cultured on ECM sheets, hydrogels, or solubilized forms derived from the three kidney regions (cortex, medulla, papilla) and compared to mesenchymal cells (MSCs) cultured under the same conditions.

The kidney is a suitable organ for studying effects of regional ECM on the resident stem cell population. A cross-sectional view of the kidney reveals three distinct regions: cortex, medulla, and papilla (FIG. 1A), with each region displaying its unique structure, function, and composition, and residing in environments with very different osmolalities and oxygen tensions.

The cortex contains renal corpuscles, and the associated convoluted and straight tubules, collecting tubules and ducts, and contains an extensive vascular network. The medulla is arranged into pyramids, and characterized by straight tubules, collecting ducts, and the vasa recta, a specialized capillary system involved in the concentration of urine. At the apex of each medullary pyramid, where the collecting ducts converge and empty into the renal calyx, is the papilla. The renal papillae contain a putative population of adult stem cells that remains quiescent after the development is complete and is mobilized again during injury. This stem cell may be isolated and expanded in culture, making the kidney an excellent model to study interactions between the native stem cell population and the matrix derived from distinct regions within the organ.

ECM materials according to various embodiments of the present disclosure are useful to grow, maintain, or differentiate organ- or region-specific cells in culture. Various embodiments of the present disclosure are useful for: in vitro three-dimensional culture and testing of cells on acellular scaffolds, in sponges, or in hydrogels; in vitro culture and testing of cells with culture medium supplemented with matrix solution; in vitro coating (adsorption) of matrix solution to cell culture flask/dish to increase attachment, growth; in vitro guided differentiation of embryonic stem cells or induced pluripotent stem cells into organ-specific cells; in vivo injection/delivery of therapeutic cells, drugs, or other soluble factors via hydrogel; in vivo implantation of acellular scaffolds with or without cells for tissue/organ regeneration studies.

The present disclosure describes a method to derive regionalized ECM biomaterials, for example, for stem cell culture. Such materials include acellular scaffolds, sponges, hydrogels, solutions, fibers (e.g., electrospun fibers) or bio-ink (e.g., printable bio-ink). According to various embodiments of the present disclosure, materials are provided in various physical forms including various sized sheets and solubilized forms. According to various embodiments, ECM biomaterials are derived from various tissues including adrenal gland, amnion, bladder, blood vessel, bone, brain, breast, cartilage, chorion, connective tissue, esophagus, eye, fat, heart, kidney, large intestine, larynx, ligament, liver, lung, lymph node, microvasculature, muscle, omentum, ovary, pancreas, placenta membrane, prostate, skin, small intestine, smooth muscle, spinal cord, spleen, stomach, tendon, testis, thymus, umbilical cord, uterus, or Wharton's Jelly. In some embodiments, region-specific ECM biomaterials are derived from a corresponding region in a source organ, for example, from the cortex, medulla or papilla of a kidney. Tissues sources include human and animal; fetal, juvenile, and adult; healthy, diseased, and transgenic.

Described below are the regionally specific effects of kidney ECM on the growth and metabolism of kidney stem cells, how these effects depend on the preservation of ECM structure vs. only composition, and extension of these effects to exogenous (non-kidney) stem cells, such as mesenchymal stem cells (MSCs).

The methods and systems presented herein may be used for creating ECM biomaterials for stem cell culture from various tissues, including region-specific kidney extracellular matrix hydrogels. The disclosed subject matter is particularly suited for creating region-specific hydrogels for the growth of stem cells, such as kidney stem cells (KSCs), and mesenchymal stem cells (MSCs).

According to embodiments of the present disclosure, native tissue matrix is used to cause region-specific effects on the growth of KSCs and mesenchymal stem cells (MSCs). To this end, hydrogels are derived from kidney regions including the cortex, medulla and papilla.

According to an exemplary method, kidneys are procured and immediately frozen and prepared for sectioning. Frozen blocks are then sectioned longitudinally into thin (200 μm-1 mm) slices showing the entire cross-section of the kidney. The cortex, medulla, and papillae of the kidney are then dissected and separated from the thin slices prior to decellularization.

The tissues are decellularized using a 4-step method consisting of 0.02% trypsin (2 hr), 3% Tween-20 (2 hr), 4% sodium deoxycholate (2 hr), and 0.1% peracetic acid (1 hr). Each step is followed by deionized water and 2× PBS washes. In some embodiments, each region is decellularized by serial washes in 0.02% trypsin, 3% Tween, 4% deoxycholic acid, and 0.1% peracetic acid solutions followed by enzymatic digestions.

Following decellularization, the ECMs are snap frozen in liquid nitrogen, pulverized using a mortar and pestle, and then lyophilized to obtain a fine powder. Lyophilized ECM powder is digested using pepsin and hydrochloric acid for 48 hours at room temperature. The resulting digest is re-constituted into a hydrogel by increasing the ionic strength and the pH of the solution using PBS and NaOH.

The results are highly specific hydrogels composed of the native extracellular matrix surrounding native cells. They may be used to grow and maintain tissue-specific cells in culture. In some embodiments, cells are cultured on the hydrogels. In other embodiments, cells are cultured in media supplemented with digested ECM. Metabolic activity, image analysis and DNA quantification may be performed.

In one embodiment, kidney stem cells isolated from the papilla are maintained by culturing the cells in papilla derived ECM hydrogels in vitro. Hydrogels may also be used as an injectable therapeutic platform for the delivery of drugs and/or cell therapy to an injured kidney or to guide the differentiation of embryonic stem cells or induced pluripotent stem cells into kidney specific cells for renal tissue engineering applications.

KSCs cultured in the presence of papilla ECM show higher metabolic activity and lower DNA content when compared to whole kidney, cortex and medulla ECM, an effect not observed using MSCs. Thus, the hydrogels derived from the native kidney ECM stimulate the parent KSCs but not the MSCs. Region specific kidney ECM affects the growth and metabolism of KSCs. Region-specific ECM thus provides a suitable substrate for cultivation and delivery of stem cells and their derivatives.

According to various embodiments of the present disclosure, ECM is extracted from organs and tissues including the adrenal gland, amnion, bladder, blood vessel, bone, brain, breast, cartilage, chorion, connective tissue, esophagus, eye, fat, heart, kidney, large intestine, larynx, ligament, liver, lung, lymph node, microvasculature, muscle, omentum, ovary, pancreas, placenta membrane, prostate, skin, small intestine, smooth muscle, spinal cord, spleen, stomach, tendon, testis, thymus, umbilical cord, uterus, or Wharton's Jelly. Organs/tissues are procured, prepared for sectioning, frozen, then sectioned into thin slices. In some embodiments, the slices are about 200 μm to about 1 mm thick. Organ regions, sub-sections, or anatomical features of interest are further dissected and separated prior to decellularization. In various exemplary embodiments, region-specific tissues are extracted from the kidney cortex, medulla, or papilla; the lung airways or parenchyma; the esophageal endomucosa or muscularis externa; or the heart ventricle or atrium.

Tissue sections are decellularized by the introduction of one or more of deionized water, hypertonic salines, enzymes, detergents, and acids. In an exemplary embodiment, lobar liver sections are decellularized by 0.02% trypsin (120 min), 0.5% Ethylenediaminetetraacetic acid (EDTA)(30 min), 3%Tween-20, (120 min), 8mM 3-[(3-cholamindoproyl)dimethlammoniol-1-propanesulfonate (CHAPS)(120 min). Each step is followed by deionized water and hypertonic (2x) phosphate-buffered saline (PBS) washes. Exemplary embodiments for various organs and tissues of human and animal origin are provided in Table 1.

TABLE 1

| Organ | Step 1 | Step 2 | Step 3 | Step 4 |
|---|---|---|---|---|
| Bladder | Trypsin | EDTA | Tween-20 | EDTA |
| | 0.02% | 0.5% | 3% | 0.5% |
| | 120 min | 30 min | 120 min | 30 min |
| Bone | 0.5M HCl | Tween-20 | EDTA | CHAPS |
| | 12 hours | 3% | 0.5% | 8mM |
| | | 120 min | 30 min | 120 min |
| Esophagus | Trypsin | EDTA | Tween-20 | EDTA |
| (mucosa) | 0.02% | 0.5% | 3% | 0.5% |
| | 120 min | 30 min | 120 min | 30 min |
| Esophagus | Trypsin | EDTA | Tween-20 | EDTA |
| (smooth | 0.02% | 0.5% | 3% | 0.5% |
| muscle) | 360 min | 30 min | 360 min | 30 min |
| Heart | Trypsin | EDTA | Tween-20 | EDTA |
| | 0.02% | 0.5% | 3% | 0.5% |
| | 60 min | 30 min | 120 mim | 30 min |
| Kidney | Trypsin | EDTA | Tween-20 | EDTA |
| | 0.02% | 0.5% | 3% | 0.5% |
| | 120 in | 30 min | 120 min | 30 min |
| Liver | Trypsin | EDTA | Tween-20 | CHAPS |
| | 0.02% | 0.5% | 3% | 8mM |
| | 120 mm | 30 min | 120 mm | 120 min |
| Lung | Trypsin | EDTA | Tween-20 | EDTA |
| | 0.02% | 0.5% | 3% | 0.5% |
| | 120 min | 30 min | 120 min | 30 min |
| Muscle | Trypsin | EDTA | Tween-20 | EDTA |
| | 0.02% | 0.5% | 3% | 0.5% |
| | 60 min | 30 min | 120 min | 30 min |

| Organ | Step 5 | Step 6 | Step 7 |
|---|---|---|---|
| Bladder | Sodium Deoxycholate, | DNase I | Peracetic Acid |
| | 4% | 0.2 mg/mL | 0.1% |
| | 120 min | 120 min | 30 min |
| Bone | | | |
| Esophagus | Sodium Deoxycholate, | DNase I | Peracetic Acid |
| (mucosa) | 4% | 0.2 mg/mL | 0.1% |
| | 120 min | 120 min | 30 min |
| Esophagus | Sodium Deoxycholate, | DNase I | Peracetic Acid |
| (smooth | 4% | 0.2 mg/mL | 0.1% |
| muscle) | 360 min | 120 min | 30 min |
| Heart | Sodium Deoxycholate, | DNase I | Peracetic Acid |
| | 4% | 0.2 mg/mL | 0.1% |
| | 120 min | 120 min | 30 min |
| Kidney | Sodium Deoxycholate, | DNase I | Peracetic Acid |
| | 4% | 0.2 mg/mL | 0.1% |
| | 120 min | 120 min | 30 min |
| Liver | | | |
| Lung | Sodium Deoxycholate, | 0 DNase I | Peracetic Acid |
| | 4% | 0.2 mg/mL | 0.1% |
| | 120 min | 120 min | 30 min |
| Muscle | Sodium Deoxycholate, | DNase I | Peracetic Acid |
| | 4% | 0.2 mg/mL | 0.1% |
| | 120 min | 120 min | 30 min |

Following decellularization, resulting materials are terminally sterilized and biopsied according to desired scaffold size. In some embodiments, the scaffold is sized to fit in the wells of a standard microtiter plate, for example a 6-, 12-, 24-, 48-, or 96-well plate.

In some embodiments, following decellularization, an ECM solution is produced. The decellularized material is snap frozen in liquid nitrogen, pulverized using a mortar and pestle, milled, and lyophilized to obtain a fine ECM powder. In some embodiments, the ECM powder is digested using 1 mg/mL pepsin and 0.1 M hydrochloric acid for more than 1 hour at room temperature. The resulting digest is neutralized, frozen, and thawed to obtain ECM solution.

In some embodiments, ECM powder is further processed to form an ECM sponge. ECM powder is digested using 1 mg/mL pepsin and 0.1 M hydrochloric acid for less than 24 hours at room temperature. The resulting digest is subjected to repeated cycles of high-speed centrifugation (5,000 rpm) and vortexing. The resulting material is transferred to a mold of desired dimensions and lyophilized. The resulting sponge can be sectioned, re-sized, or rehydrated. In some embodiments, the sponge is sized to fit in the wells of a standard a microtiter plate, for example a 6-, 12-, 24-, 48-, or 96-well plate.

In some embodiments, ECM solution is ECM solution is re-constituted into a hydrogel by increasing the ionic strength and the pH of the solution using PBS and sodium hydroxide.

Composition and Gelation Properties of Decellularized Kidney ECM

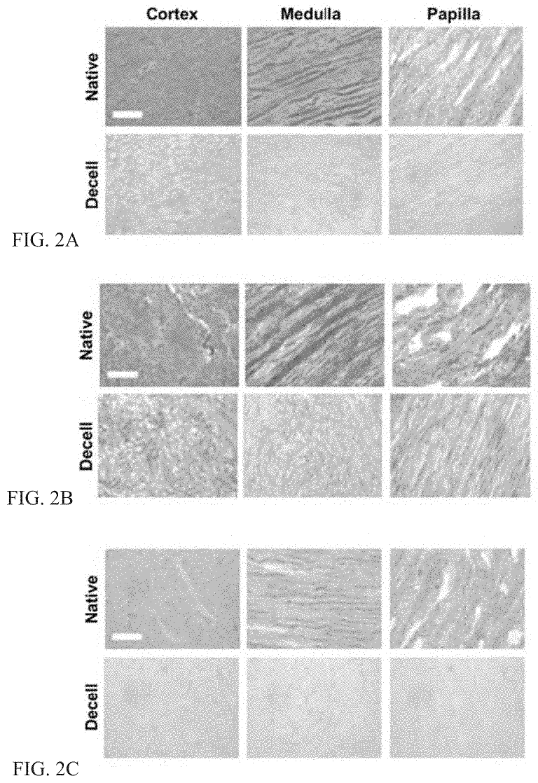

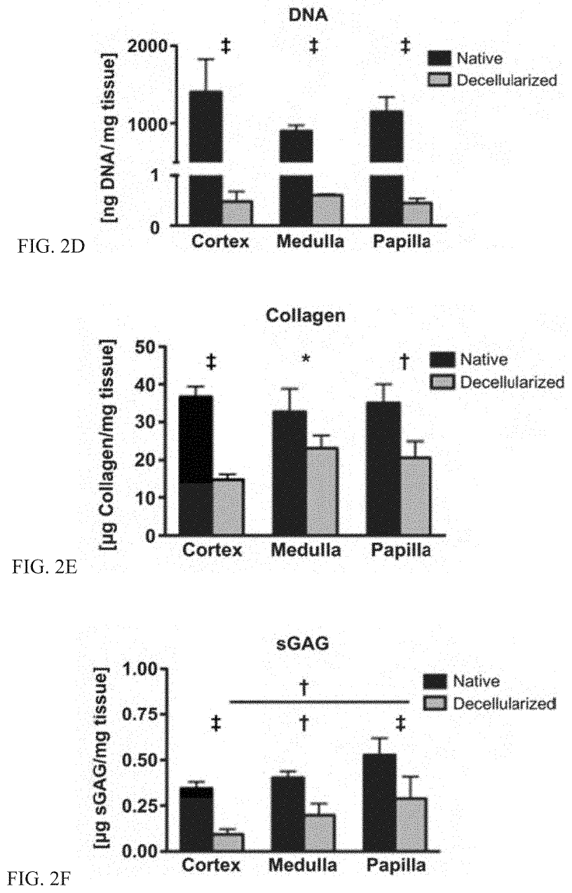

Referring to FIGS. 2A-2C, removal of cellular material and preservation of ECM in decellularized kidney regions is illustrated. Histology confirms decellularization with preservation of matrix proteins in kidney regions. In FIG. 2A: H&E stain shows the absence of cell nuclei. In FIG. 2B: trichrome stain shows the preservation of collagen (blue), and in FIG. 2C: Alcian Blue stain shows loss of proteoglycans (light blue). In FIG. 2D: DNA quantification indicates >99% removal of nuclear material after decellularization. In FIG. 2E: collagen quantification shows comparable retention of collagen among kidney regions. In FIG. 2F: sulfated glycosaminoglycan (sGAG) quantification indicates papilla retains significantly more sGAG than cortex.

Decellularization of kidney regions (cortex, medulla, papilla) by a four step method (trypsin, Tween 20, sodium deoxycholate, peracetic acid) resulted in the removal of >99% nuclear material as shown by H&E staining and DNA quantification (FIGS. 2A, 2D). Collagen content of decellularized kidney regions was reduced in all three regions, and most significantly in the cortex (FIGS. 2B, 2E). A similar trend was observed with sulfated glycosaminoglycans (sGAG) content (FIGS. 2C, 2F). Histological sections of kidney regions stained with H&E (FIG. 2A), Trichrome (FIG. 2B), and Alcian Blue (FIG. 2C) show complete removal of cellular nuclei with some preservation of ECM structure and distribution of remaining collagen (blue) (FIG. 2B) and glycosaminoglycans (blue) (FIG. 2C).

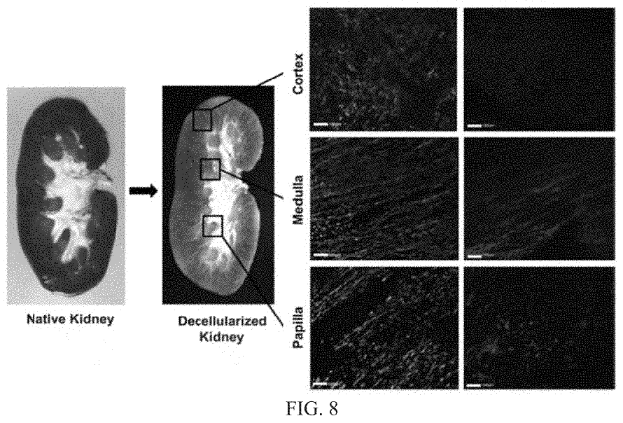

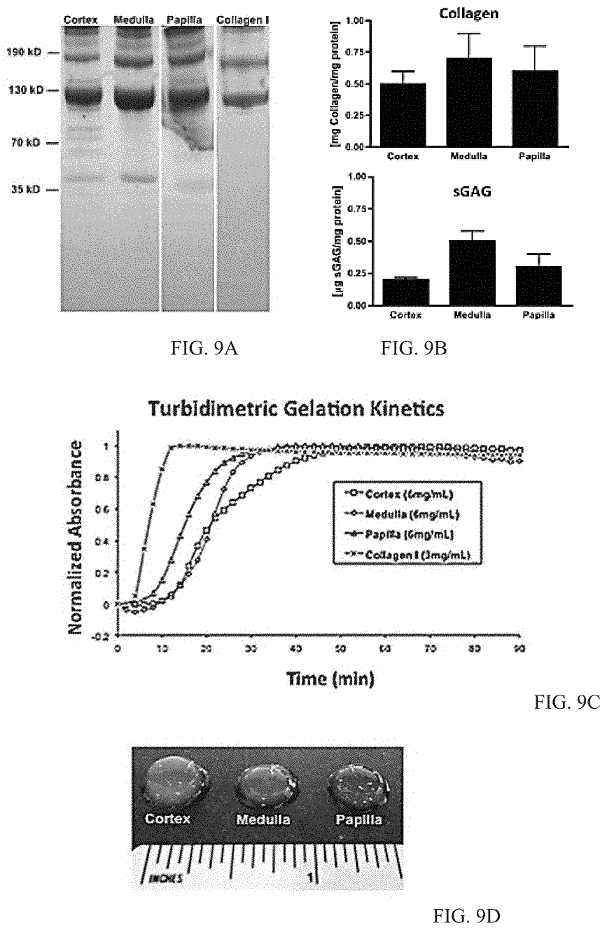

Electrophoresed kidney region ECM digests and purified collagen I showed major bands at similar locations, indicating that collagen I is a large component of the kidney region ECM digests, with other bands distinct from pure collagen I (FIG. 9A). In the digested (solubilized) ECM from kidney regions, the amounts of collagen per unit ECM protein were comparable among the three regions, while the amount of sGAG per unit ECM protein was lower for the cortex region (FIG. 9B). The measurements of gelation kinetics showed sigmoidal curves for ECM derived from all kidney regions and the collagen I hydrogel, with kidney hydrogels having delayed kinetics relatively to collagen I (FIG. 9C). Among the kidney hydrogels, the time for gelation increased from papilla to medulla and cortex. Polymerized kidney region hydrogels had similar macroscopic appearance (FIG. 9D).

Ultrastructure of Native and Decellularized Kidney ECM

Referring to FIGS. 3A-3B, ultrastructure of native and decellularized kidney regions is illustrated. In FIG. 3A, scanning electron microscopy at 350× reveals differences in ECM topography between kidney regions before and after decellularization. In FIG. 3B, transverse sections of decellularized papilla at 100× and 350× indicate that tubular ultrastructure is preserved in the KSC niche after decellularization.

Native and decellularized kidney regions were imaged via SEM to investigate preservation of the ultrastructure after decellularization (FIG. 3). Increased magnification at 350× reveals large differences in the native structures of the ECM in various regions of the kidney as well as distinct topographical differences retained in decellularized kidney regions (FIG. 3A). A transverse section of decellularized papilla reveals preservation of tubular ultrastucture in the KSC niche (FIG. 3B).

Collagen IV and Fibronectin in Native and Decellularized Kidney ECM

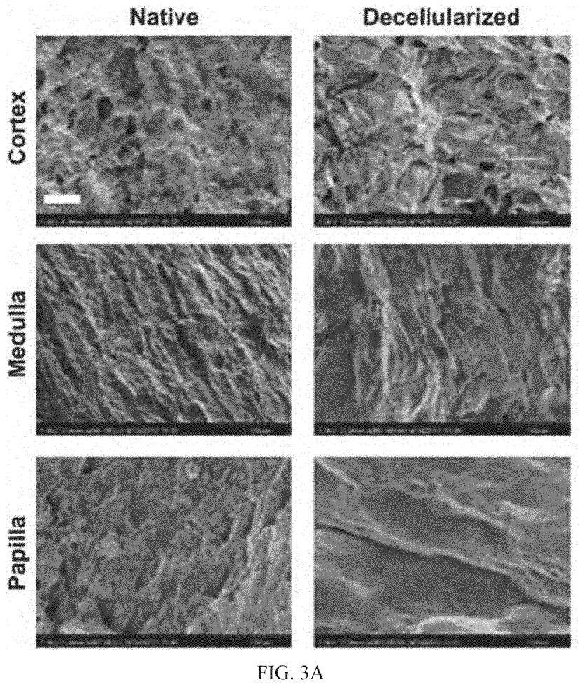

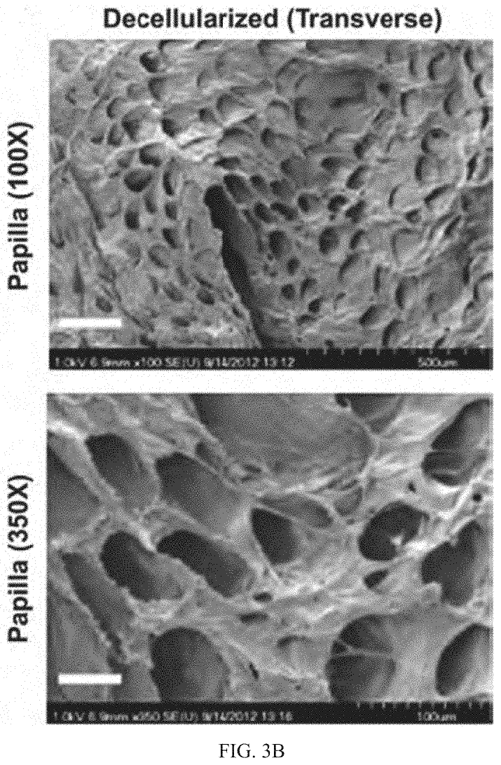

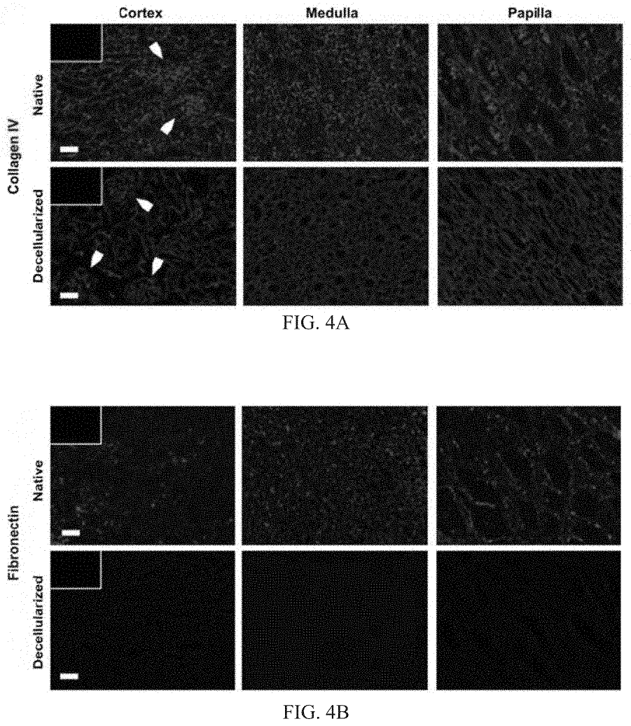

Referring to FIGS. 4A-4B, collagen IV and fibronectin native and decellularized kidney regions are illustrated. Immunostaining reveals in FIG. 4A significant retention of collagen IV in the basement membrane of decellularized kidney regions, including preservation of glomerular structures in decellularized cortex (arrows), and in FIG. 4B depletion of fibronectin in kidney regions after decellularization.

Native and decellularized kidney regions were immunostained to reveal the amounts and distributions of collagen IV and fibronectin in kidney regions before and after decellularization (FIGS. 4A-4B). Immunostaining for collagen IV indicates a significant amount of collagen IV is retained after decellularization as well as the retention of renal corpuscular structures in the cortex (FIG. 4A). Immunostaining for fibronectin indicates a significant loss of fibronectin after decellularization (FIG. 4B).

DNA and Metabolic Activity of KSCs in Solubilized Kidney ECM

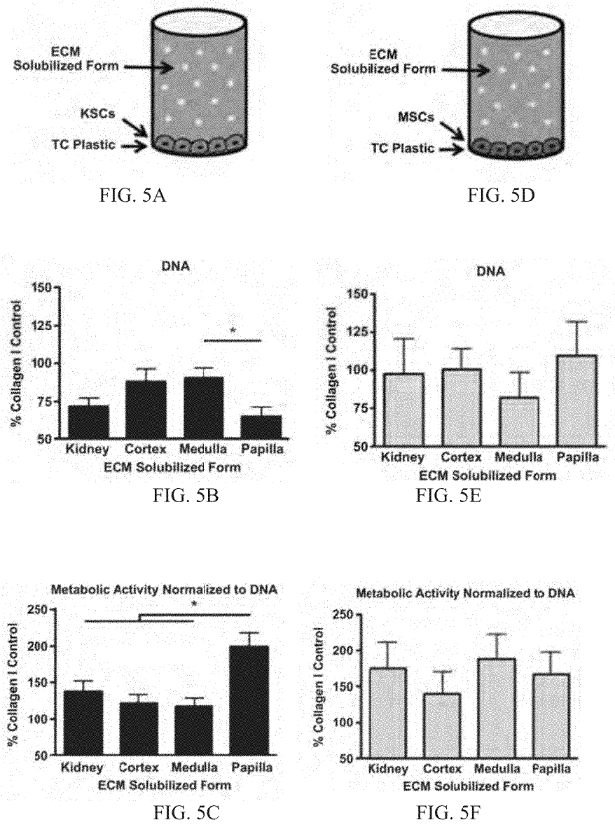

Referring to FIGS. 5A-5F, DNA and metabolic activity of KSCs and MSCs in the presence of solubilized regionally specific kidney ECM is illustrated. In FIG. 5A, KSCs were cultured on tissue culture plastic in the presence of solubilized whole organ or regional kidney ECM. In FIG. 5B, DNA quantification of KSCs revealed less DNA in the presence of solubilized papilla ECM than in the presence of solubilized ECM from cortex, medulla, or whole kidney. In FIG. 5C, KSCs cultured in the presence of solubilized papilla ECM were significantly more metabolically active than KSCs cultured in the presence of solubilized ECM from cortex, medulla, or whole kidney. In FIG. 5D, MSCs cultured on tissue culture plastic in the presence of solubilized whole kidney and kidney region ECM. In FIG. 5E, DNA quantification revealed no significant differences between MSCs cultured in the presence of whole organ or regional solubilized kidney ECM. In FIG. 5F, metabolic activity of MSCs showed no differences between MSCs cultured in the presence of whole organ or regional solubilized kidney ECM.

KSCs and MSCs were cultured on tissue culture plastic in media supplemented with solubilized ECM derived from the three regions or the whole kidney. DNA and metabolic activity were measured and expressed relatively to the corresponding values measured for cells grown in media supplemented with purified solubilized collagen I (FIGS. 5A, 5D). The number of KSCs in cultures with solubilized papilla ECM was significantly lower than in the solubilized ECM from any other region or the whole kidney (FIG. 5B), indicating that papillary ECM suppresses cell cycling. The whole kidney ECM showed an intermediate value between the ECMs of the three regions. Metabolic activity per unit DNA indicates that KSCs grown in solubilized papilla, although fewer in number, were significantly more metabolically active than KSCs grown in solubilized whole kidney, cortex, or medulla ECM.

No significant differences in metabolism were observed between KSCs in solubilized cortex and medulla ECM (FIG. 5C). MSCs in solubilized kidney region ECM showed no significant differences in DNA or metabolic activity (FIGS. 5E, 5F).

DNA, Metabolic Activity, and Phenotype of KSCs on Regional Kidney ECM Hydrogels

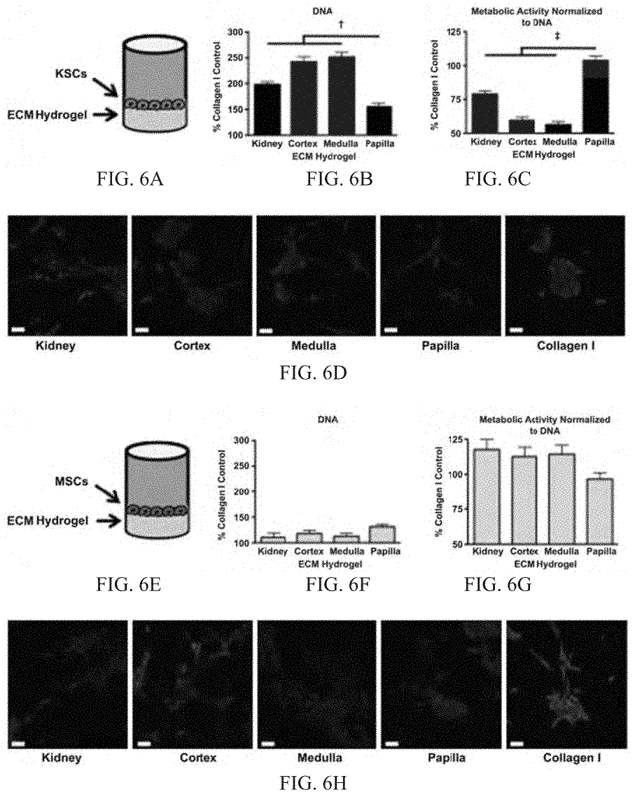

Referring to FIGS. 6A-6H, metabolic activity, DNA content, and rhodamine-phalloidin/DAPI staining of KSCs and MSCs on regional kidney ECM hydrogels are illustrated. In FIG. 6A, KSCs seeded onto ECM hydrogels and cultured for 48 hrs. In FIG. 6B, DNA quantification of KSCs shows that papilla hydrogel contained significantly fewer cells than EXM derived from other kidney regions or the whole kidney. In FIG. 6C, metabolic activity normalized to DNA reveals that KSCs are significantly more metabolically active on papilla ECM hydrogel than on ECM hydrogels from other kidney regions or the whole kidney. In FIG. 6D, confocal imaging of KSCs on regional kidney ECM hydrogels and collagen I hydrogel shows longitudinal cell alignment in kidney ECM hydrogels but not in collagen I. In FIG. 6E, MSCs seeded onto ECM hydrogels and cultured for 48 hrs. In FIG. 6F, DNA quantification of MSCs also shows no significant differences between regional kidney hydrogels. In FIG. 6G, metabolic activity per unit DNA for MSCs was comparable for all ECM hydrogels, derived regionally or from the whole kidney. In FIG. 6H, confocal imaging of MSCs on kidney region hydrogels and collagen I shows similar cell morphology in all kidney ECM hydrogels and collagen I hydrogel. Scale bars: 50 μm.

KSCs and MSCs were seeded at equal densities on decellularized kidney ECM hydrogels (FIGS. 6A, 6E), cultured for 48 hrs, assayed for DNA and metabolic activity, and data were normalized to those measured for collagen I hydrogel. DNA quantification of KSCs revealed significant differences between whole kidney, cortex, medulla, and papilla regions, with papilla hydrogel again yielding significantly fewer KSCs (FIG. 6B). The whole kidney hydrogel showed an intermediate value approximating an average for the three regions. Equal initial seeding densities resulted in significantly more KSCs on kidney region ECM hydrogels than MSCs after 48 hrs (FIGS. 6B, 6F).

Metabolic activity per unit DNA again indicated that KSCs on papilla ECM hydrogel were significantly more metabolically active than were KSCs on whole kidney, cortex or medulla ECM hydrogel. No significant differences were observed in metabolism between KSCs on cortex and medulla hydrogels (FIG. 6C). Morphology of KSCs on whole kidney and regional kidney hydrogels appeared consistent (FIG. 6D). No significant differences in DNA, metabolic activity, or morphology were observed for MSCs on kidney region ECM hydrogels (FIGS. 6F, 6G, 6H).

DNA, Metabolic Activity, and Phenotype of KSCs on Regional Kidney ECM Sheets

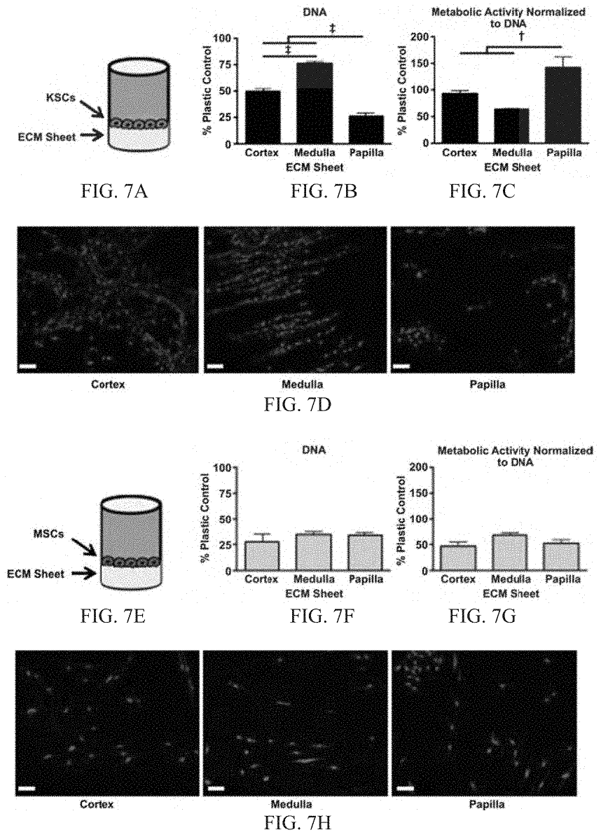

Referring to FIGS. 7A-7H, metabolic activity, DNA content, and rhodamine-phalloidin/DAPI staining of KSCs and MSCs on region-specific decellularized kidney ECM sheets are illustrated. In FIG. 7A, KSCs seeded onto ECM sheets and cultured for 48 hrs. In FIG. 7B, DNA quantification reveals that papilla ECM contained significantly fewer cells, and that medulla contained significantly more cells than either cortex or papilla. In FIG. 7C, metabolic activity per unit DNA indicates that the fewer KSCs on papilla are significantly more metabolically active than KSCs on cortex or medulla ECM. In FIG. 7D, rhodamine-phalloidin/DAPI staining shows clear differences in morphology, orientation, and structure formation between KSCs on cortex, medulla, and papilla ECM sheets. KSCs on cortex show star-like morphology with random orientation, whereas KSCs on medulla exhibit elongated morphology with significant aligning and the formation of tubular structures. KSCs on papilla show clusters with periodic rounded morphology. In FIG. 7E, MSCs are seeded onto ECM sheets and cultured for 48 hrs. In FIG. 7F, DNA quantification shows no significant differences in MSC cultures on ECM from different kidney regions. In FIG. 7G, metabolic activity per unit of DNA reveals no differences in metabolic activity of MSCs. In FIG. 7H, rhodamine-phalloidin/DAPI staining shows consistency in MSC number and phenotypes in ECM from all kidney regions. Scale bars: 50 μm.

KSCs and MSCs were seeded at equal densities on decellularized kidney ECM sheets (FIGS. 7A, 7E), cultured for 48 hrs, assayed for DNA and metabolic activity, and data were normalized to those measured for cells grown on tissue culture plastic.

DNA quantification of KSCs cultured on decellularized ECM sheets revealed significant differences between cortex, medulla, and papilla regions, with papilla ECM again yielding the fewest KSCs (FIG. 7B). Metabolic activity per unit DNA confirms that KSCs on papilla were significantly more metabolically active per cell than were KSCs on either cortex or medulla (FIG. 7C). No significant differences in metabolism between KSCs on cortex and medulla ECM sheets were observed.

1009911n addition to differences in cell number, distinct morphologies and orientation of KSCs were observed in cortex, medulla, and papilla ECM (FIG. 7D). No significant differences in DNA, metabolic activity, or morphology were observed for MSCs on kidney region ECM sheets (FIGS. 7F, 7G, 7H). Significantly more KSCs were observed on decellularized kidney region ECM when compared to MSCs after 48 hrs (FIGS. 7B, 7F).

Structure Formation by KSCs on Kidney ECM Sheets

Referring to FIG. 8, live/dead and rhodamine/phalloidin staining of KSCs and MSCs on regionally specific decellularized kidney region ECM sheets are illustrated. KSCs cultured decellularized kidney ECM from different regions display significantly different cell number, morphology, orientation, and structure formation at 48 hours. KSCs on cortex sheets show star-like morphology in random aggregations, whereas KSCs on medulla exhibit elongated morphology with significant aligning and formation of tubular structures. KSCs on papilla show some aligning but also periodic rounded morphology not seen in cortex or medulla. Rhodamine-phalloidin staining highlights significant differences in KSC morphology and orientation between kidney regions after 7 days. The corresponding cultures of MSCs show no differences cell number, morphology, or alignment. Scale bars: 100 μm.

KSCs were seeded at equal densities onto ECM sheets derived from decellularized kidney regions (cortex, medulla, and papilla), cultured for 48 hrs or 7 days, and imaged. KSCs showed clear differences when cultured on ECM sheets from different kidney regions in cell morphology, orientation, and structure formation already by 48 hrs of cultivation (FIG. 8). On decellularized cortex sheets, KSCs consistently showed star-like morphology and regional aggregations (FIG. 8, top). On decellularized medulla, KSCs displayed elongated morphology, end-to-end alignment, and tubular formations distinctly not seen in decellularized cortex at 48 hrs (FIG. 8, middle). On decellularized papilla, KSCs appeared morphologically different from KSCs on cortex, with some alignment in the upper papilla similar to KSCs on medulla (FIG. 8, bottom). Additionally, KSCs with a rounded morphology were periodically observed in decellularized papilla sheets, only rarely in medulla, and not in cortex. KSCs on cortex displayed structures resembling renal corpuscles similar to those seen in native cortex H&E histological sections, while KSCs on medulla displayed many straight tubular bundles similar to the medullary rays seen in medulla H&E histological sections.

Metabolic Activity of KSCs on Whole Organ ECM

FIGS. 10A-C show kidney stem cells (KSCs) seeded onto tissue culture plastic and cultured for 48 hrs in three different forms of ECM (decellularized sheets, hydrogels, and solubilized forms as shown schematically at the top of each Figure) obtained from porcine hearts, bladders and kidneys. KSCs grown on decellularized whole kidney sheets and whole kidney hydrogel showed significantly higher metabolic activity at 48 hrs when compared to KSCs grown on bladder and heart ECM sheets and hydrogels (FIGS. 10A, 10B) when normalized to cells grown on tissue culture (TC) plastic. Furthermore, KSCs cultured on tissue culture plastic in the presence of solubilized whole kidney ECM were more metabolically active than KSCs cultured in the presence of solubilized bladder and heart ECM, with a significant difference between cells cultured in the presence of kidney and bladder ECM (FIG. 10C). These results indicate that KSCs cultured on or in the presence of whole kidney ECM in various forms—decellularized sheets, hydrogels, and solubilized forms—are significantly more metabolically active than KSCs cultured in various forms of ECM from other organs, suggesting a degree of recognition or specificity by endogenous kidney stem cells to the ECM of their native organ.

Chemotaxis (Transwell) Assay

KSCs seeded onto transwell membranes with 8 μm pores were cultured in the presence of solubilized kidney region ECM as shown schematically in FIG. 11A. KSCs cultured in the presence of solubilized papilla ECM demonstrated the least chemotaxis across the membrane, while KSCs cultured in solubilized cortex ECM demonstrated the most chemotaxis (FIG. 11B).

Discussion

Kidney stem cells cultured on whole kidney ECM were compared to ECM derived from the urinary bladder and heart to determine if there was recognition between KSCs and the ECM at the organ level. ECM from whole bladder, heart, and kidney was prepared in three different forms: decellularized sheets, hydrogels, and solubilized forms. KSCs were significantly more proliferative and metabolically active in all three forms of kidney ECM when compared to respective forms of bladder or heart ECM (FIGS. 9A-9D).

The organ specificity of KSCs according to the present disclosure demonstrates specificity of liver sinusoidal endothelial cells to liver ECM and indicates that decellularized kidney ECM sheets contain organ-specific cues. Higher KSC metabolism is observed in whole kidney ECM hydrogel and soluble ECM, where the ECM ultra-structure is absent and only a homogenous mix of digested ECM proteins (cross-linked in the hydrogel or dissolved in solution) comprises the extracellular environment (FIGS. 9B, 9C). Taken together, these data indicate that the interactions responsible for cell-matrix recognition is not limited to structural cues from decellularized matrix but also relies on signaling from small molecules or protein fragments.

A degree of kidney stem cell-matrix specificity has been shown at the organ level. Accordingly methods are provided to isolate and prepare ECM biomaterials from three distinct regions of the kidney—the cortex, medulla, and papilla—to show cell-matrix interactions at the regional level. Each region of the kidney harbors a variety of cell types and structures, including extensive networks of tubules, collecting ducts, and capillaries, necessary for filtering blood or concentrating urine. In an adult mouse kidney, label-retaining cells (KSCs) remain quiescent in the renal papilla (stem cell niche) and migrate to the site of injury following renal ischemia. Consequently, this adult kidney stem cell population may be used to investigate region-specific effects of kidney ECM on the proliferation and metabolism of KSCs.

Characterization of the ECM in native cortex, medulla, and papilla reveals significant differences in structure and composition, many of which are retained after decellularization and further processing. Following the removal of >99% nuclear material (FIGS. 2A, 2D), some ECM proteins—such as collagens I and IV, were preserved similarly across regions of the kidney (FIGS. 2B, 2E, 4A), while the amount of sGAG (FIGS. 2C, 2F) as well as overall ECM ultra-structure (FIG. 3) differed significantly across regions. In all regions, the cell adhesion molecule fibronectin was significantly depleted after decellularization (FIG. 4B).

Scanning electron micrographs of kidney region ECM showed comparable topographies between native and decellularized sections, indicating that many ultra-structural features of the ECM are retained after cells are removed. Additionally, large tubular collecting ducts approximately 50 μm in diameter are seen in decellularized papilla sections (FIG. 3B) and demonstrate distinguishing ultra-structural features of the KSC niche not found in medulla or cortex. Given the sensitivity of stem cells to their environment, such differences in matrix architecture between kidney regions may account for differences observed in KSC activity and morphology.

While structural cues account for some organ or even region-specific signaling to kidney stem cells, compositional cues from the ECM also play a role in informing KSCs about their extracellular environment. Decellularized whole mouse kidney ECM are able to direct the differentiation of embryonic stem cells into specialized cells types as well as to encourage proliferation along the basement membrane, indicating that the basement membrane or one or more of its components promotes signaling for proliferation. Differences in the composition and distribution of the basement membrane in different regions of the kidney thus account for some of the region-specific differences observed in KSC proliferation and metabolism. Further, kidney region ECM biomaterials may be used to selectively differentiate KSCs into region-specific cell types.

As shown in FIG. 7D, KSCs showed significant differences in cell number, morphology, and arrangement as a function of the region from which the ECM was derived. Such differences are not observed with MSCs (FIG. 7H), indicating that structural and/or compositional differences in the kidney ECM are recognized by KSCs. In FIG. 8, KSCs seeded on the cortex show arrangement into distinct circular shapes similar to the renal corpuscular structures seen in the immunostaining of native cortex sections (FIG. 4A). Together these data indicate that the KSCs were able to recognize the type of matrix and adopt morphology similar to the native tissue. Further, KSCs may be differentiating into one or multiple cells types in situ.

Across the ECM regions discussed above, KSCs cultured in papilla ECM consistently showed significantly lower cell number (DNA content) when compared to KSCs in cortex and medulla ECM (FIGS. 5B, 6B, 7B). Similar trends in mitochondrial activity and cell number are observed at two different time points (48 hours and 7 days) when cultured in three different forms of kidney ECM: sheets, hydrogels, and solubilized forms (FIGS. 5C, 6C, 7C). This trend indicates that the effect of the ECM on KSCs' growth and metabolic activity does not depend on the structural form of the ECM but rather on the composition. This is further supported by the trend in metabolic activity when cultured in hydrated or solubilized forms of the ECM where high metabolic activity was found in the papilla ECM and low metabolic activity in the cortex and medulla ECM.

When cultured in ECM obtained from entire kidney sections (containing cortex, medulla, and papilla), the metabolic activity was found to be within the values obtained for the individual regions, suggesting a dose effect. In addition, factors may be more readily available in the solubilized form but may still be locked into place or obscured by other proteins in an intact decellularized sheet.

One aspect of this work is the development of tissue-specific biomaterials and the potential for tissue regeneration using regionalized ECM biomaterials to direct the differentiation of reparative stem cells, for example to address renal pathologies such as diabetes or kidney failure. This approach translates into other regionalized organs as well. Cultivation of epithelial and endothelial cells on fully decellularized rat kidney scaffolds in a whole-organ perfused bioreactor results in a bioengineered kidney that produced rudimentary urine in vitro (in the bioreactor) and in vivo (following orthotopic implantation in rat). A variety of cell and tissue engineering applications may be applied in conjunction with regionalized ECMs. For example, since the renal papilla is the KSC niche, it may be used to maintain the cells in a stem-like state in vitro, while cortex and medulla ECM may be used to differentiate KSCs into other renal cell types.

Ischemic conditions in the cortex encourage mobilization, migration, and differentiation of quiescent KSCs in the papilla. The data in FIG. 11B support these findings. Solubilized factors from damaged matrix could provide chemotactic cues for KSCs, signaling them to migrate to the site of injury and differentiate into cell types for repair and regeneration. In addition, ECM hydrogels allow for production in large quantities when compared to ECM sheets (by pooling ECM from a large number of kidneys) as well as the use of ECM derived from regions that are small in size or volume, such as the renal papilla.

With regard to FIGS. 9A-9D, organ-specific effects of ECM on metabolism of kidney stem cells are illustrated. In FIG. 9A, kidney stem cells (KSCs) show significantly higher metabolic activity when cultured on decellularized ECM sheets derived from kidney as compared to either bladder or heart ECM sheets. Data are normalized to cells grown on tissue culture (TC) plastic. In FIG. 9B, KSCs cultured on whole bladder, heart, and kidney ECM hydrogels also showed significantly higher metabolic activity for kidney ECM than other organ hydrogels when normalized to cells grown on TC plastic. Data are normalized to cells grown on tissue culture (TC) plastic. In FIG. 9C, after 24-hr starvation, KSCs cultured on TC plastic showed higher metabolic activity in media supplemented with solubilized (digested) whole kidney ECM than solubilized ECM from other organs.

With regard to FIGS. 10A-10C, characterization of solubilized kidney region ECM and ECM hydrogels is illustrated. In FIG. 10A, gel electrophoresis of solubilized kidney ECM digests and collagen I. In FIG. 10B, collagen and sGAG quantification of solubilized kidney ECM. In FIG. 10C, turbidimetric gelation kinetics of kidney region hydrogels. In FIG. 10D, kidney region ECM hydrogels.

With regard to FIGS. 11A-11B, chemotaxis of KSCs in the presence of solubilized kidney region ECM. In FIG. 11A, transwell chemotaxis assay experimental set-up is illustrated. KSCs are seeded on an 8 mm porous membrane and allowed to migrate through the membrane in thepresence of different solubilized ECM chemotactic factors. In FIG. 11B, KSCs in solubilized papilla ECM showed the least amount of chemotaxis relative to KSCs cultured in solubilized cortex and medulla ECM. Solubilized cortex ECM instigated significantly more KSC chemotaxis than solubilized papilla ECM.

ECM biomaterials derived from the tissues affect the growth and metabolism of stem cells with regional specificity. Region-specific ECM may thus provide an optimal substrate for the in vitro cultivation or the delivery of therapeutic stem cells and their derivatives. The present disclosure is application to development of biomaterials or applications in tissue repair and regeneration using regionalized ECM biomaterials to deliver and direct the differentiation of reparative stem cells to address pathologies such as diabetes or kidney failure.

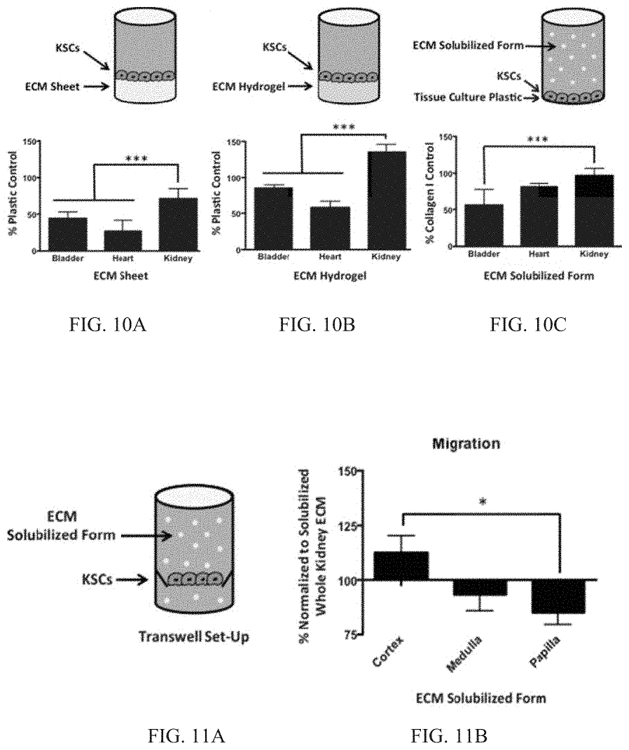

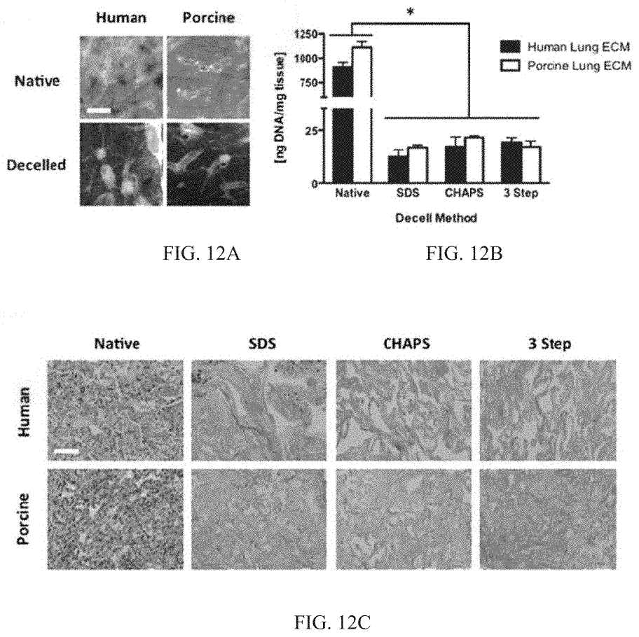

FIGS. 12A-C and 13A-D show characterization of lung ECMs using the sodium dodecyl sulfate (SDS), 3-[(3-cholamidopropyl)dimethylammonio]-1-propanesulfonate (CHAPS), and 3-step methods. Data represent mean±SE (n=9 for each group). *p<0.05.

FIGS. 12A-C show characterization of lung decellularization. FIG. 12A shows macroscopic images of human and porcine lung tissue slices before and after decellularization. Scale bar: 5 mm. FIG. 12B shows DNA quantification before and after decellularization. FIG. 12C shows hematoxylin and eosin stain of the three decellularization methods at x40. Scale bar: 50 mm. Decellularized human and porcine lung tissue slices appeared translucent, with visible conduits throughout the matrix (FIG. 12A). Decellularization consistently removed more than 95% of the nuclear material (FIG. 12B). Hematoxylin and eosin staining showed no discernible nuclei FIG. 12C). Histologic analysis revealed retention of small anthracotic aggregations (black regions) in some human lung sections; these regions were generally avoided during imaging.

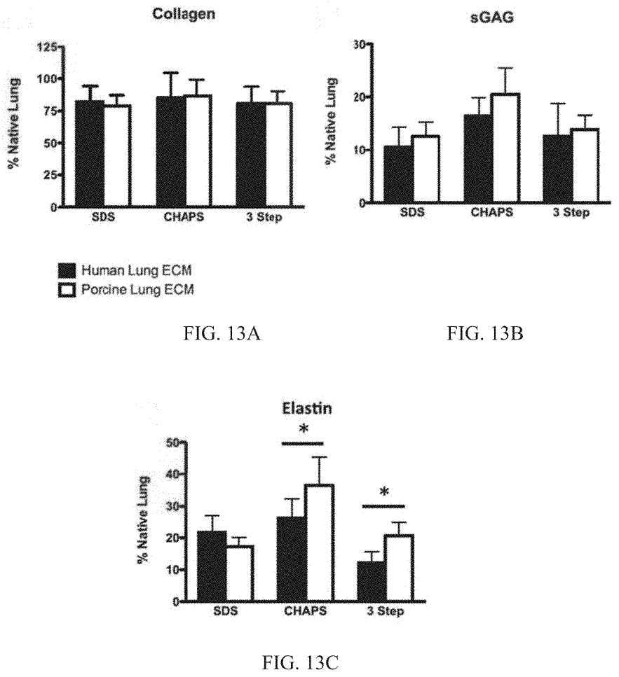

FIGS. 13A-C show characterization of collagen, sulfated glycosaminoglycans, and elastin in lung ECMs. FIG. 13A shows graphs of collagen content. FIG. 13B shows graphs of sulfated glycosaminoglycan content. FIG. 13C shows graphs of elastin content.

Both human and porcine LECM retained 80% of total collagen regardless of the decellularization method used (FIGS. 13A, 13D). However, all three methods substantially decreased the amount of sGAG (FIGS. 13B, 13D), with no apparent differences between the human and porcine LECM. There was a significant difference between human and porcine LECM in the amount of elastin retained after decellularization using CHAPS and the three-step method (FIG. 13C). These quantitative results were consistent with histologic staining using Masson's Trichrome (collagen, blue), Alcian Blue (sGAG, blue), and Van Gieson's (elasticfibers, black).

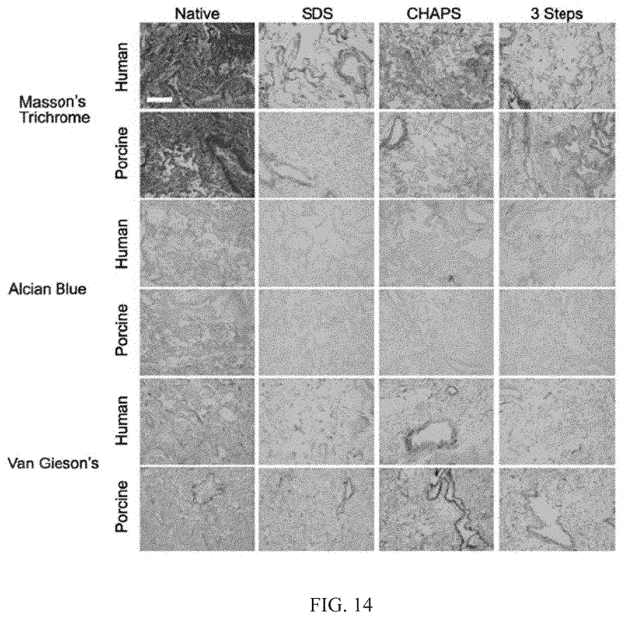

FIG. 14 shows histologic evaluation of human and porcine lung tissues after decellularization by the three methods. Masson's trichrome (collagen, blue), Alcian blue (sGAG, blue), and Van Gieson's (elastic fibers, black) staining of decellularized human and porcine lung tissue at x20 objective. Scale bar: 100 mm. (CHAPS=3-[(3-cholamidopropyl) dimethylammonio]-1-propanesulfonate; SDS=sodium dodecyl sulfate.)

FIGS. 15A-D shows distributions of extracellular matrix proteins of native tissue compared to ECMs prepared by the three methods. Representative immunohistochemical stains are shown for (Group A images) collagen IV, (Group B images) laminin, (Group C images) fibronectin, and (Group D images) elastin. All images were acquired with a x10 objective. Scale bar: 100 mm. Collagen IV was found in the basement membrane of both native and decellularized human and porcine tissue with no apparent differences among decellularization methods (FIG. 15A). By contrast, laminin and fibronectin were not well retained in either porcine or human LECM, with fibronectin most depleted by the SDS method (FIGS. 15B, 15C). Finally, there was higher retention of elastin for CHAPS compared with the SDS and three-step methods (FIG. 15D).

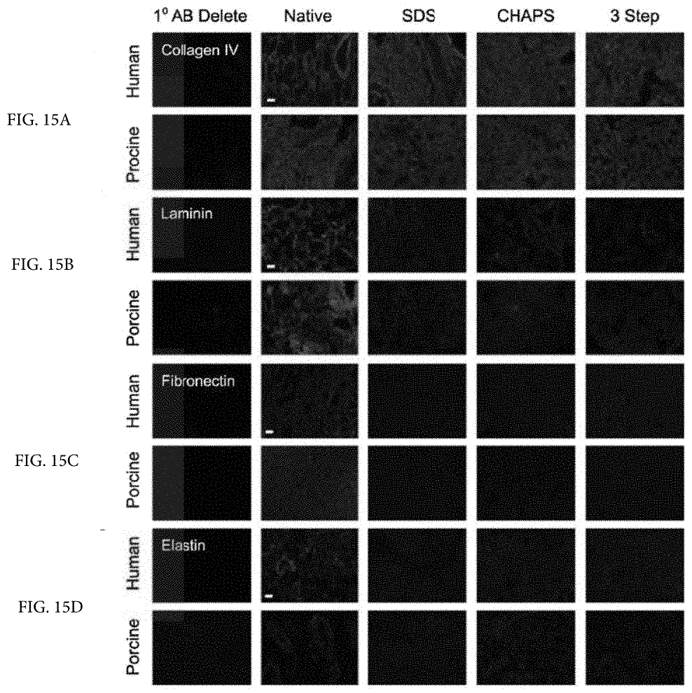

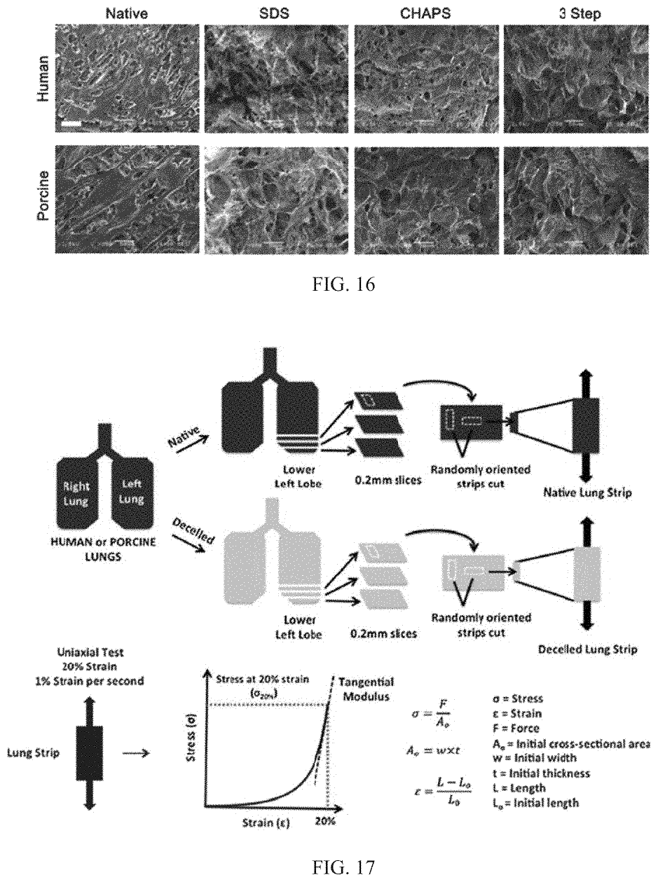

The ultrastructural morphologies of human and porcine LECM before and after decellularization were similar, as evidenced by electron microscopy (FIG. 16). Representative scanning electron micrographs are shown for all experimental groups. Scale bar: 50 mm. Native lung slices showed smooth surfaces that were disrupted after decellularization, resulting in a more fibrillar structure and a rougher topographic profile. Decellularization using SDS showed the most fibrillary ultrastructure of all the methods. Overall, there were no major differences in ultrastructural morphology between human and porcine LECM.

FIG. 17 illustrates schematically preparation of decellularized human and porcine LECMs for mechanical characterization. Slices were obtained from the lower left lobes of human and porcine lungs and decellularized by three different methods. Samples were randomly obtained from the transverse sections of the lung and tested in uniaxial tensile strain.

Significant differences were observed in the mechanical testing (FIGS. 18A, 18B). Representative uniaxial stress-strain curves for (FIG. 18A) human and (FIG. 18B) porcine lung extracellular matrix (ECM) in their native state and after decellularization. Linear correlation was detected between the elastin content and (FIG. 18C) the maximum stress and (FIG. 18D) tangential modulus at 20% strain for all decellularization methods of decellularized human and porcine lung ECM. Data represent mean±SE (n=9). Both the tangential modulus and peak stress experienced at a 20% strain were higher for human than for porcine LECM. CHAPS decellularization resulted in the highest tangential modulus and peak stress for human and porcine LECM. The most compliant LECM was observed for the three-step and SDS methods in the human and porcine LECM, respectively. Because CHAPS-decellularized tissue showed the best retention of LECM structure, hMRC-5s, hSAECs, and human adipose-derived mesenchymal stem cells hMSCs were cultured on CHAPS-decellularized human and porcine LECM.

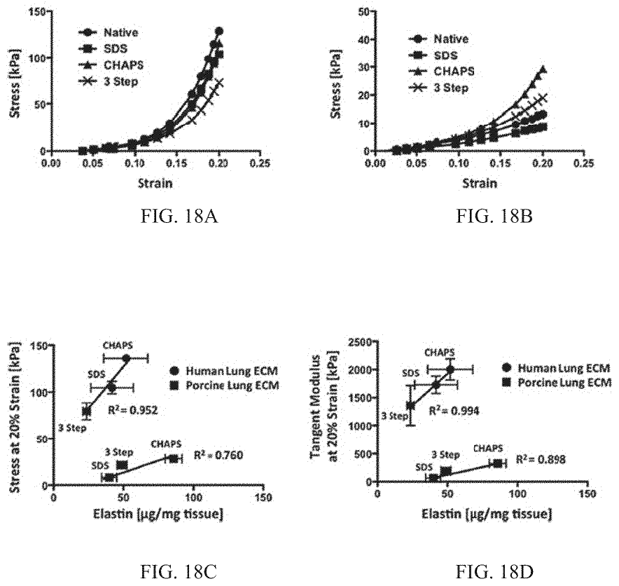

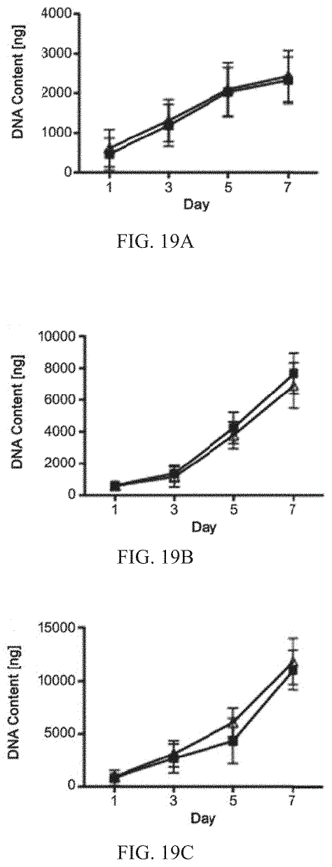

FIGS. 19A-C show growth curves of 3 human cell types on lung scaffolds decellularized by CHAPS. Growth curves for 7 days culture of three different types of cells on CHAPS-decellularized human (solid squares) and porcine (Δ) lung extracellular matrix (ECM). FIG. 19A shows human lung fibroblasts (hMRC-5s). FIG. 19B shows human small airway epithelial cells (hSAECs). FIG. 19C shows Human adipose-derived mesenchymal stem cells (hMSCs). Data represent mean±SE (n=9).

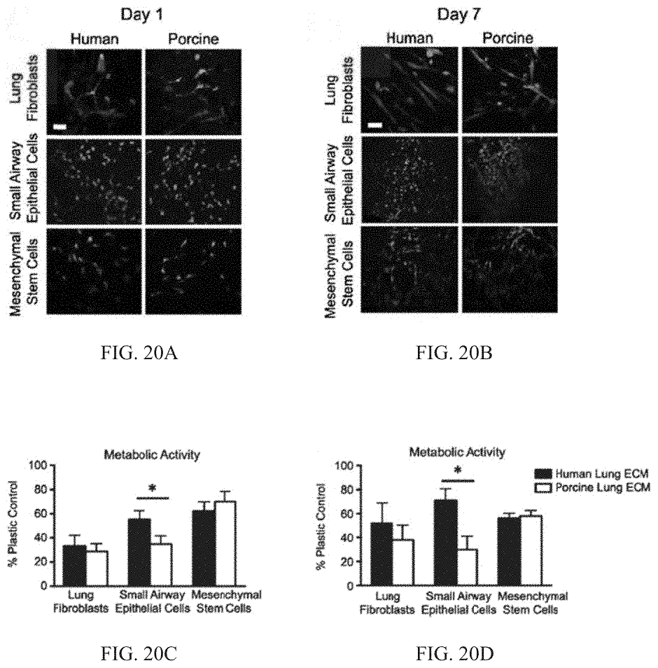

FIGS. 20A-D illustrate viability and metabolic activity of three human cell types on lung scaffolds decellularized by CHAPS. FIGS. 20A and 20B show Cell viability (live cells stained green for calcein-AM, dead cells stained red for ethidium homodimer-1) for human lung fibroblasts (hMRC-5s), human small airway epithelial cells (hSAECs), and human adipose-derived mesenchymal stem cells (hMSCs) after 1 and 7 days of culture on decellularized lung matrix. Scale bar: 40 μm. FIGS. 20C and 20D show metabolic cell activity measured after 1 and 7 days of culture.

All cell types proliferated at comparable rates over a 7-day period (FIGS. 19A-19C), were fully viable over 7 days of culture (FIGS. 20A, 20B), and had comparable metabolic rates (FIGS. 20C, 20D). However, hSAECs were more metabolically active in human than in porcine LECM over 7 days of culture (FIGS. 20C, 20D).

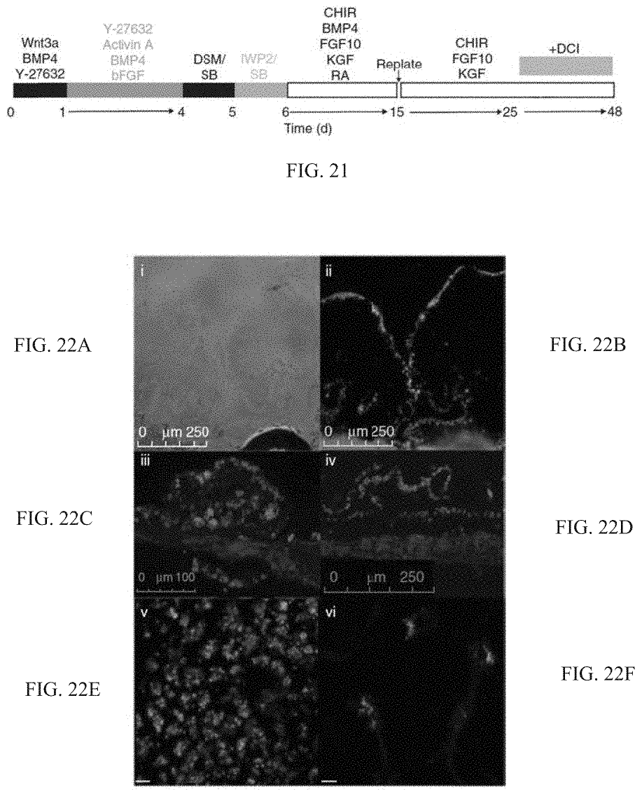

FIG. 21 shows a protocol for culturing cells to examine the effect of adding dexamethasone, 8-bromo-cAMP and isobutylmethylxanthine (DCI), factors that induce alveolar maturation in fetal mouse lung explants and enhance surfactant protein expression in mouse ESC—derived lung progenitors.

We cultured the cells from day 15 on in the presence of decellularized slices of human lung. Initially, NKX2.1+p63+ cells were seen to be adhering to the matrix (FIGS. 22A-22B). Subsequently, cells overgrew the matrix slices and showed positive staining for SP-B (FIGS. 22C-22D)) and uptake of BODIPY-SP-B at day 48 (FIG. 22E). The morphology of the BODIPY SP-B+cells was very similar to that seen using two-photon microscopy after uptake of BODIPY-SP-B in live mouselung (FIG. 22F). These data show the ability of the NKX2.1+p63+ cells to attach to native lung matrix, grow and express distinct functional properties of pulmonary cells, an important feature for potential applications in which lung progenitors are used to treat lung disease.

FIGS. 22A-22F show confocal fluorescence micrograph images of aspects of the generation of airway and lung epitheal cells from human pluripotent stem cells after being seeded on decellularized human lung matrix. FIGS. 22A-22B show expression of p63 and NKX2.1 at day 25 of cultures of RUES2 cells seeded on slices of decellularized human lung matrix at day 15 of the protocol. Initially, NKX2.1+p63+ cells were seen to be adhering to the matrix. Subsequently, cells overgrew the matrix slices and showed positive staining for SP-B (FIGS. 22C-22D) and uptake of BODIPY-SP-B at day 48 of culture with DCI (FIG. 22E Scale bar, 5 μm). FIG. 22F shows the morphology of mouse ATII cells as observed by two-photon microscopy of live mouse lung after instillation of BODIPY-SP-B. Scale bar, 5 μm.

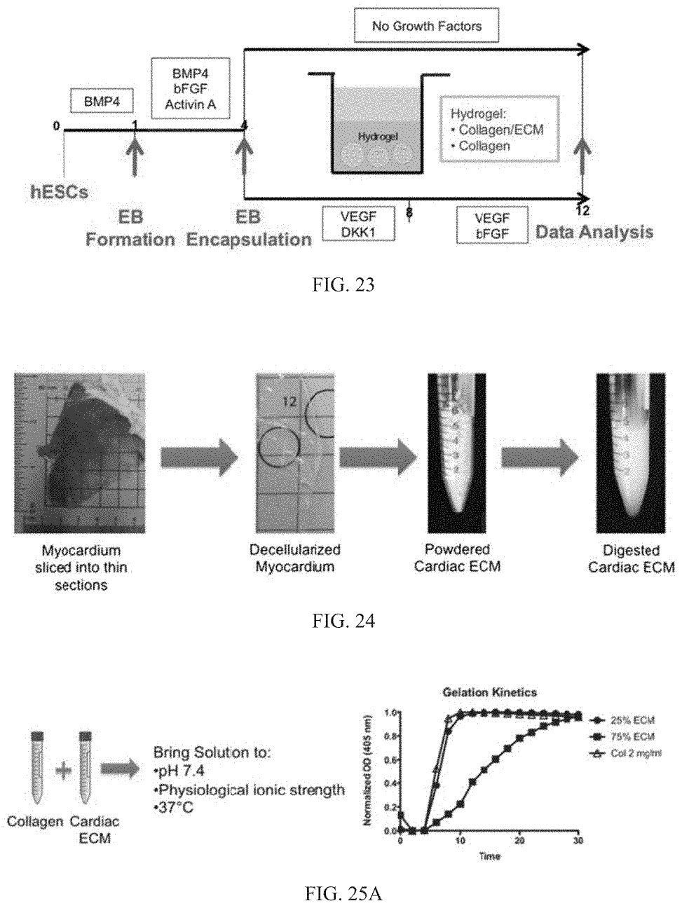

FIG. 23 shows a model system for cultivation of human embryonic derived cardiomyocytes encapsulated in hydrogels. Human embryonic stern cells (hESCs) undergo the formation of a primitive-streak-like population from day 1 to day 4, the induction and specification of cardiac mesoderm from day 4 to day 8, and the expansion of the cardiovascular lineage from day 8 on. On day 4, embryonic bodies (EBs) were encapsulated in three types of hydrogels, each cultured with or without supplementation of growth factors. Media were changed every 4 days.

FIG. 24 shows schematically the derivation of cardiac ECM digests. Frozen porcine hearts were thawed and sliced into thin sections. The sections were decellularized, crushed, and lyophilized to obtain a cardiac ECM powder. The powder was digested using pepsin to obtain cardiac ECM digests.

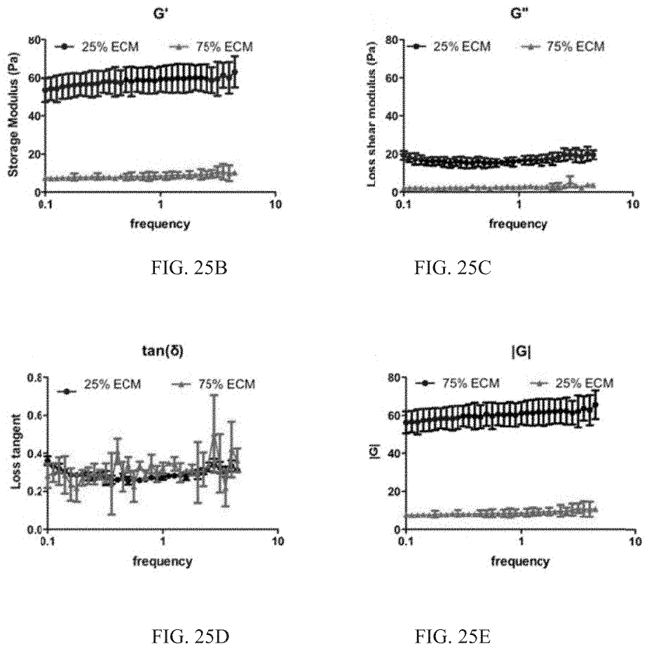

FIGS. 25A-E show gelation and mechanical properties of cardiac hydrogels. FIG. 25A shows a schematic of hydrogel preparation and the measurements of gelation kinetics based on turbidity. Cardiac ECM digest was neutralized at 4° C. and brought to 37° C. inside a spectrophotometer, and the turbidity was monitored over time. The normalized gelation kinetics is shown as a function of time for pure collagen, 75% ECM, and 25% ECM hydrogels. FIGS. 25B-C show storage and loss moduli for 75% ECM and 25% ECM hydrogels, respectively. The storage modulus increased when the frequency increases 0.1 to 5 Hz by ˜16% for 25% ECM and by ˜38% for 75% ECM The loss modulus of 25% ECM hydrogel first decreased with an increase in loading frequency from 0.1 to 0.5 Hz and then continued to increase till 5 Hz, resulting in a bell-shaped curve. Both the storage and loss shear moduli changed significantly with the ECM concentration. FIG. 25D shows graphs of the loss tangent 75% ECM and 25% ECM hydrogels. When the ECM content decreases from 75-5%, both storage and loss shear moduli increased by over six times, while the phase shift angle δ or loss tangent remained unchanged (˜17 and ˜0.3, respectively, for 75% and 25% hydrogels. FIG. 25E shows the dynamic shear modulus as a function of frequency. At 1 Hz, a rate that is close to the physiological heart beating rate, the dynamic shear modulus was 61.5+5.9 Pa for 75% ECM gel and 8.6+1.5 Pa for 25% ECM gel. For comparison, the dynamic shear modulus of 2 mg/ml pure collagen is 25 Pa, Mechanical data are presented as mean±SD. Morphology and Cellularity of Hydrogel-Encapsulated EBs

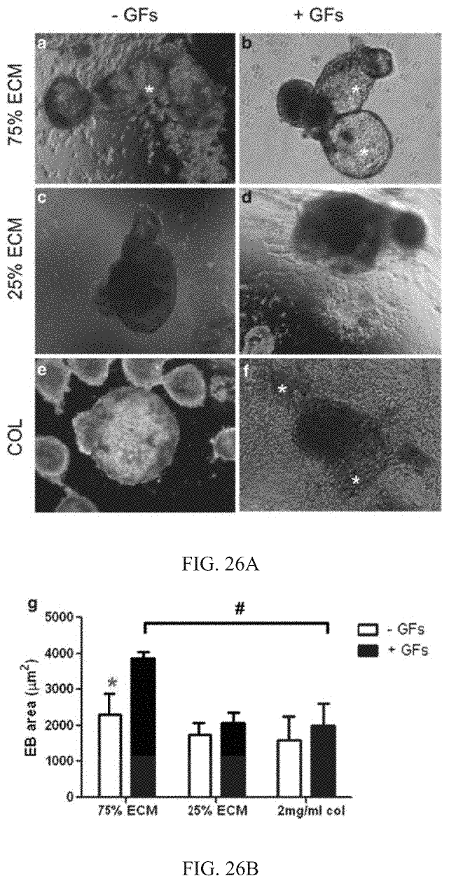

FIGS. 26A-B show the characterization of EBs. Images a-f of FIG. 26A show the morphology of EBs cultured in hydrogels with and without supplemental growth factors. FIG. 26A s EB sizes under different culture conditions determined using the images in three separate EB differentiation experiments. Analysis of EB morphology and cellularity was performed at day 12 after the initiation of cell differentiation. FIG. 26A shows representative images for EBs in hydrogels for all six experimental groups (three hydrogels, with and without supplementation of growth factors). In both 75% ECM groups, EBs appeared heterogenous in size. (images a, b, stars) and tended to group together. In contrast, EBs in collagen retained their original spherical shape. Representative images of cardiomyocytes in collagen gels with and without growth factors are shown in images e, f, respectively. Data represent mean±SD. In the Figures, GF=growth factors; ECM extracellular matrix; COL=2 mg/ml collagen gel.

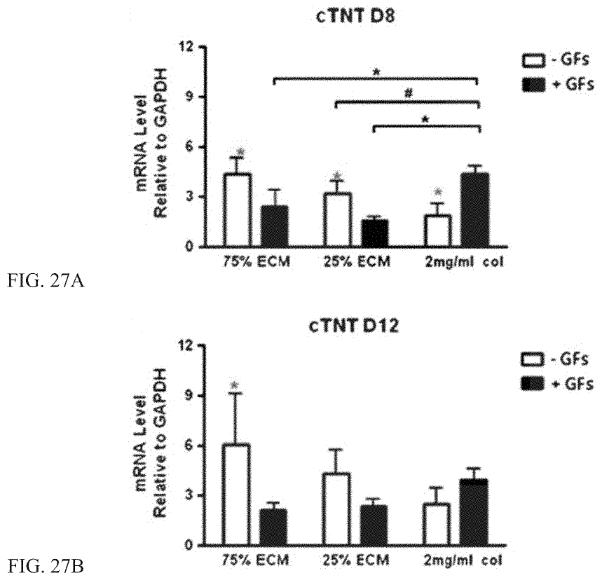

FIGS. 27A-B show levels of expression of cardiac troponin T (cTnT). Data are shown for three groups of hydrogels with and without supplemental growth factors. GAPDH was used as an endogenous control. Expression in blend gels was measured at day 8 (27A) and day 12 (27B). Data represent mean±SD. To evaluate cardiac differentiation of hESCs in three dimensional hydrogels with and without supplemental growth factors, we assessed the mRNA expression of cardiac troponin T (cTnT), a widely used mature cardiac marker, at day 8 and day 12 of differentiation. At day 8, the expression level of cTnT was significantly higher in both the 25% and 75% ECM hydrogel groups without the supplemental growth factors. In contrast, the supplementation of growth factors to the collagen group increased expression of cTnT. Interestingly, the expression level of cTnT in the 75% ECM group without supplemental factors was comparable to that in collagen group with supplemental growth factors (FIG. 27A). By day 12, the effects of supplemental growth factors in 75% ECM group remained significant, in contrast to 25% ECM and collagen groups (FIG. 26B). Also, by day 12 of culture, the expression of cTnT became comparable for all hydrogel groups with supplemental growth factors.

Contractile Function of Differentiating Cells

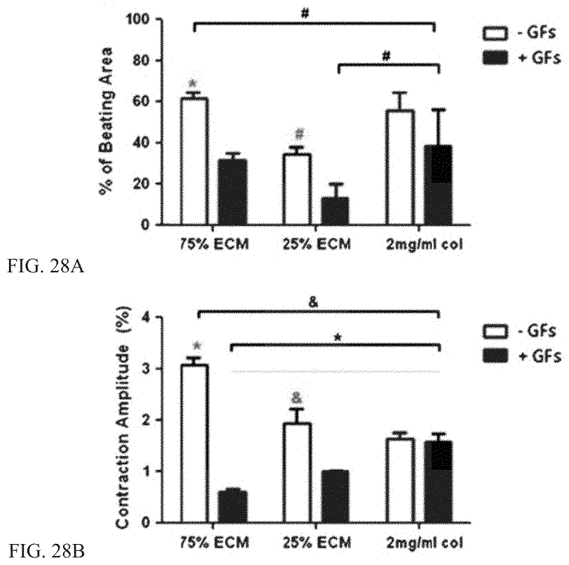

FIGS. 28A-B show characterization of contractile behavior. Data are shown for three groups of hydrogels with and without supplemental growth factors. Contractile behavior of cells, a functional indication of cardiac differentiation, was observed in all experimental groups, with and without supplemental growth factors. At day 12 of differentiation, the percent of beating area in both ECM groups was higher without than with the addition of supplemental factors (61.8±6.7% vs. 30.8±3.2% for the 75% ECM gel; 34.8±9.7% vs 13.2±7.1% for the 25% ECM gel) and highest for the 75% ECM group among all hydrogel groups (FIG. 28A). Similarly, the contraction amplitude of cells within EBs cultured in both ECM groups was significantly higher without than with supplementation of growth factors; such effect was not observed for collagen hydrogel group (FIG. 28B). EBs in 75% ECM hydrogels showed markedly higher contraction amplitudes compared with either 25% ECM or collagen hydrogel (3.09±0.17% vs. 1.94±0.55 vs. 1.58±0.13%). Taken together, the measurements of contractile behavior showed that the 75% ECM hydrogel group without supplemental growth factors outperformed all other groups.

Expression of Cardiac Markers

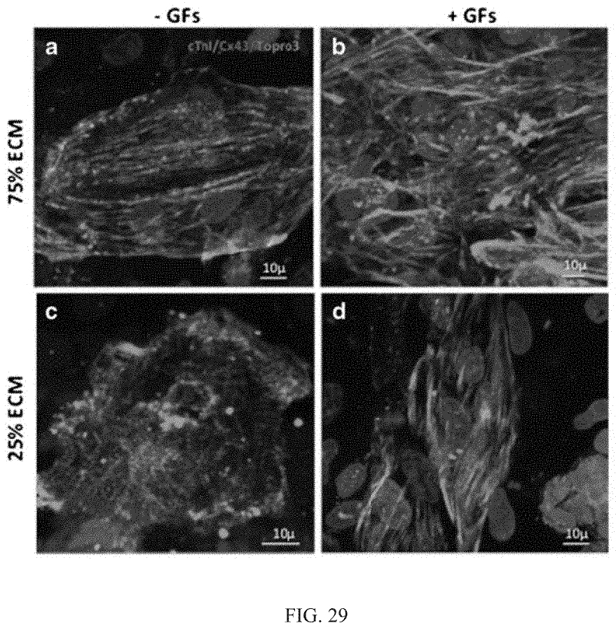

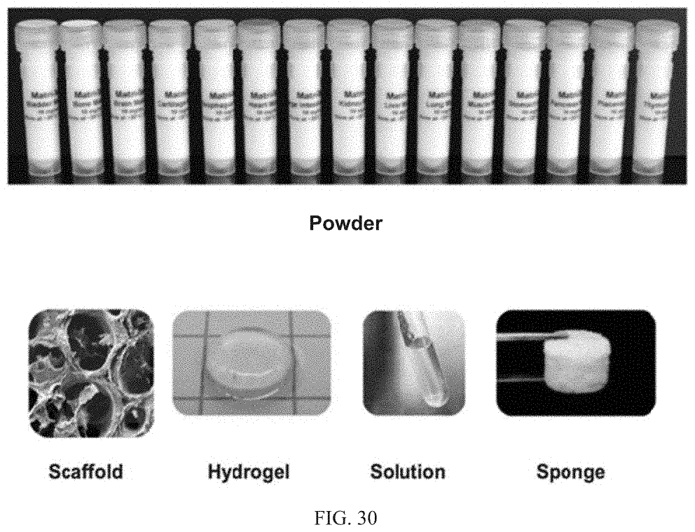

Cardiac-specific immunofluorescence staining using distinct marker cardiac troponin I (cTnI) and Cx43 was performed. FIG. 29 shows confocal images of cardiac markers in human ESC derived cardiomyocytes cultured in different hydrogels. Bar, 10 μm, FIG. 29 shows that the cells in hydrogels express positive staining for cTnI. Only cells in hydrogel without growth factors demonstrated the typical striation patterns indicative of mature sarcomeres (images a, c). Cells in 75% ECM revealed the most organized distribution of Cx43, localized both at the cell periphery and at the perinuclear sites, as compared with scattered localization of Cx43 in cells cultured in 25% ECM (image a). The expression of Cx43 was also upregulated when cells were cultured without the supplementation of growth factors (images a,c). In comparison, in both 75% and 25% ECM hydrogels with supplemental growth factors, Cx43 demonstrated limited expression among the ctnI-positive cells (images b,d), with clusters of other types of cells expressing most of Cx43 (image d). Notably, the striation structure occupied only 20% of the entire cTnI positive area in 75% ECM hydrogels supplemented with growth factors (image b). These results indicate that native ECM promotes the differentiation of hESCs into cardiac lineage and that further supplementation of growth factors did not enhance the efficiency of the differentiation.

FIG. 29 shows confocal images of cardiac markers in human ESC derived cardiomyocytes cultured in different hydrogels. Confocal images of cardiac marker, troponin I (cTnI, green) and connexin 43 (Cx43, red) staining patterns in human ESCderived cardiomyocytes cultured in different hydrogels in the absence or presence of growth factors. Bar, 10 μm.

### EXAMPLES

Decellularization

Porcine bladders, hearts, and kidneys were procured from Yorkshire pigs (65-70 kg) immediately following euthanasia, excess tissue was trimmed, and the blood and debris removed with water. The organs were stored at −80° C. for at least 24 hrs, thawed and then sliced into <2 mm thin cross-sections.

Human lungs and porcine lungs were removed, and similar regions of the lower left lobes were used for decellularization and characterization. Human lungs rejected for transplantation were procured from the New York Organ Donor Network under a protocol approved by the Institutional Review Board at Columbia University. The porcine lungs were obtained from Yorkshire pigs weighing 40 to 50 kg after the animals were used in another research study not affecting lungs and were killed under a protocol approved by the Columbia University Institutional Animal Care and Use Committee. Upon removal, the lungs were cleared of blood and immediately frozen at −80C until use, then partially thawed and sectioned to 2-mm-thick sheets.

Cross-sections from the middle third of the kidney were separated into cortical, medullary, and papillary regions. Whole kidneys and kidney regions were decellularized using a modification of a previously established method. Briefly, the slices were washed with 2× phosphate-buffered saline (PBS) for 15 min, followed by 2 hrs of 0.02% trypsin, 2 hrs of 3% Tween-20, and 2 hrs of 4% sodium deoxycholate treatment. After each step, kidney sections were washed with 2× PBS for 15 min. Kidney ECM slices were treated for 1 hr with 0.1% peracetic acid and subjected to alternating sterile 1× PBS and distilled H₂O washes.

Lung sections were washed with 2× phosphate buffered saline (PBS) for 15 minutes and placed in a series of decellularization solutions on an orbital shaker, using 1 of the following three protocols:

(1) SDS: Four 2-hour washes with 1.8 mM SDS, each followed by dH2O (5 min) and 2× PBS (15 minutes).

(2) CHAPS: Four 2-hour washes with 8 mM CHAPS, each followed by dH2O (5 minutes) and 2× PBS (15minutes).

(3) Three-step method: 2-hour wash with 3% Tween-20, 2-hour wash with 4% sodium deoxycholate, 1-hour wash with 0.1% peracetic acid.

All slices were then subjected to alternating 1× PBS and distilled H₂O washes (2 of each). Then, 7-mm-diameter discs of decellularized tissue were punched with a biopsy punch under sterile conditions and used in experiments. Pen/strep (5% each) was added to all solutions to eliminate native and pathologic bacteria from the LECM.

For cardiac ECM studies, the porcine hearts were thawed, and then myocardium (left and right ventricles) was sliced into thin (<1 mm) sections and decellularized. Briefly, the slices were initially washed with 2× phosphate-buffered saline (PBS) for 15 min followed by 2 h of 0.02% trypsin. 2 h of 3% Triton X, and 2 h of 4% deoxycholate treatment. After each step, heart slices were washed with 2× PBS for 15 min. Finally,the slices were treated for 1 h with 0.1% peracetic acid, subjected to alternating 1× PBS and dH2O washes, cut into smaller pieces, and snap-frozen in liquid nitrogen. Frozen pieces were pulverized using a mortar and pestle and lyophilized.

Preparation of Native Cardiac ECM Hydrogels

The lyophilized decellularized cardiac powder was digested. Briefly, 1 g of lyophilized ECM powder was mixed with 0.1 g of pepsin in 0.01 N HCl. The solution was allowed to digest for 48 h at room temperature under constant stirring. The final solution was aliquoted and stored at −80° C. until use. Gels were prepared by mixing collagen and cardiac ECM with 10× PBS and 0.1 N NaOH at 4° C. to yield a final concentration of 4 mg/ml with the appropriate cardiac ECM to collagen ratio. PBS, 10×, and 0.1 N NaOH were used to bring the collagen and cardiac ECM digest stock solutions to neutral salt concentration and pH.

Histological Analysis

Native and decellularized tissue samples were fixed in formalin, embedded in paraffin, sectioned at 5 μm thickness, stained with hematoxylin and eosin, Masson's Trichrome, Alcian Blue, or Von Gieson's stains and imaged using an Olympus IX81 microscope at 10×.

DNA Quantification of Decellularized Tissue

DNA content of decellularized tissue was quantified using Quanti-iT PicoGreen dsDNA Assay kit (Invitrogen) according to the manufacturer's instructions. Briefly, tissue samples were weighed, digested overnight with Proteinase K in TEX buffer at 56° C. (kidney) or papain at 60° C. (lung) and mixed with PicoGreen reagent. Fluorescence emission was measured at 520 nm with excitation at 480 nm, and DNA was quantified using a standard curve.

ECM Characterization

Collagen content of kidney or lung ECMs was determined using the Sircol collagen assay kit (Biocolor). Samples were digested in 0.1 mg pepsin/mL overnight at room temperature (25° C.), and the Sircol assay was performed according to the manufacturer's instructions.

Sulfated glycosaminoglycan (sGAG) content of kidney or lung ECMs was determined using the 1,9-dimethylene blue (DMB) dye binding assay. Samples were digested in 125 μg papain/mL overnight at 60° C. sGAG content was quantified by mixing ECM digest samples with DMB dye in a 1:5 ratio and reading spectrophotometric absorbance at 595 nm and 540 nm. The difference in absorbance at these wavelengths was used with a chondroitin-6-sulphate standard curve to quantify sGAG content.

Pepsin digests of the regional kidney ECM and collagen I (BD, Biosciences) were electrophoresed on 7.5% polyacrylamide gels (BioRad) under reducing conditions (5% 2-mercaptoethanol). The proteins were visualized with Coomassie Brilliant Blue (BioRad) and imaged by scanning the polyacrylamide gel.

Elastin was quantified by use of the Fastin elastin assay kit (Biocolor) according to the manufacturer's instructions. Tissue samples were weighed, and watersoluble α-elastin was extracted via three hot 0.25-M oxalic acid extractions, which were combined for each sample (35 mg tissue per 1 mL solution).

Scanning Electron Microscopy (SEM)

Native and decellularized sections of kidney or lung ECMs were fixed in formalin, rinsed in 70% EtOH, frozen, lyophilized, and gold-coated (5 nm thickness). Sections were imaged on a Hitachi S-4700 FE-SEM with accelerating voltage 2.5 kV or JEOL JSM 5600LV SEM.

Immunohistochemical Staining

Sections of ECMs were fixed in formalin for 30 min, embedded in paraffin, cut to 5 p.m (kidney) or 8 μm (lung), and mounted on slides. Sections were deparaffinized and subjected to boiling citrate buffer (pH 6.0) for 16 minutes for antigen retrieval, and blocked with 10% Normal Goat Serum in PBS for 2 hrs at room temperature. Primary antibody staining was performed for 2 hrs at 4° C. using the following primary antibodies and dilutions: collagen IV (Rb pAb to Coll IV, ab6586) diluted 1:200 and Fibronectin (Rb pAb to Fibronectin (ab23750)) diluted 1:200. For all stains, the secondary antibody (Goat pAb to Rb IgG (ab98464)) was diluted 1:200 and incubated for 1 hr at room temperature. Sections were mounted in Vectashield Mounting Medium with DAPI, cover slipped, and imaged with an Olympus IX81 microscope at 10×.

Mouse Kidney Stem Cells

Mouse kidney stem cells (KSCs) were obtained from mouse kidneys as previously described, cultured in Dulbecco's Modified Eagle Medium (DMEM) with high glucose supplemented with 10% fetal bovine serum (FBS) and 1% penicillin/streptomycin under standard culture conditions (37° C. and 5% CO2).

Mouse Mesenchymal Stem Cells

Mouse mesenchymal stem cells (MSCs) were obtained from Texas A&M Health Science Center College of Medicine Institute for Regenerative Medicine and cultured in Iscove's Modified Dulbecco's Medium (IMDM) supplemented with 10% FBS, 10% horse serum, and 1% penicillin/streptomycin under standard culture conditions (37° C. and 5% CO₂).

Human Lung Fibrolasts

Human lung fibroblasts (hMRC-5s) were obtained from ATCC (www.atcc.org) and cultured in Dulbecco's modified Eagle medium (DMEM) with 10% fetal bovine serum (FBS) and 1% pen/strep under standard culture conditions. The hSAECs were obtained from Dr. Gao and Dr. Minna (Dallas, Tex.) and were cultured in small airway growth media from ATCC. The hMSCs were obtained from Dr. Gimble (Baton Rouge, La.) and cultured in DMEM/F12 (1:1) with 10% FBS and 1% pen/strep.

Preparation of ECM Sheets, ECM Hydrogels, and Solubilized ECM

Decellularized whole organ and regional kidney slices were either perforated with a 7 mm biopsy punch into sheets or snap-frozen in liquid nitrogen. Sheets were stored in 1× PBS at 4° C. until use while frozen pieces were pulverized into a fine powder using a mortar and pestle, and lyophilized for 24 hrs. Lyophilized ECM powder was digested as previously described. Briefly, 1 g of lyophilized ECM powder was mixed with 0.1 g pepsin (Sigma, about 2500 U/mg) in 0.01 M HCl. The solution was allowed to digest for 48 hrs at room temperature (25° C.) under constant stirring. Final digests were aliquoted and stored at −80° C. until use. The soluble ECM was obtained by neutralizing ECM stock digests and added to cell culture media directly (Typically 1 mg dry ECM/ml medium). Hydrogels were prepared as previously described by mixing ECM stock digests with 1× PBS, 10× PBS, and 0.1 M NaOH to yield a hydrogel with a final concentration of 6 mg/mL at 4° C.

Encapsulation of Cardiac-Derived hESCs in Hydrogel

Six experimental groups were formed, by culturing EBs in three different drogels (collagen; 25% ECM in collagen; 75% ECM in collagen), with and without supplemental growth factors. At day 4 of differentiation, EBs formed from hESCs were removed from culture, washed with IMDMmedium supplemented with antibiotics, and resuspended (0.5 million cells/ml) in either plain culture medium or medium supplemented with growth factors (VEGF and DKK1 that are normally supplemented at days 4-8 stage of culture). Fifty microliters of EB suspension was added to I ml of pure collagen (2 mg/ml) solution, or to 1 ml of 75% ECM/25% collagen and 25% ECM/75% collagen hydrogel solutions. The collagen and ECM solutions were sterilized prior to encapsulation under UV light in a laminar flow hood for 30 min at 4° C. Twenty-four-well plates were coated with 50 μl of the indicated hydrogel solution and incubated at 37° C. for 30 min to allow for gelation before encapsulating the EBs. After gentle mixing, 250 μl of each EB/gel mixture was transferred (in quadruplicate) to coated wells and likewise incubated at 37° C. for 30 min. Fresh medium was then added to each sample and changed at day 8 and day 12 of culture in accordance with the differentiation protocol. Hypoxic conditions were maintained throughout the first 12 days of culture.

Solubilized Mitogenicity Assay

KSCs and MSCs were seeded on tissue culture plastic (TCP) at 2.5×10⁴ cells/mL, cultured for 24 hrs in media supplemented with 10% FBS, and starved for 24 hrs in media containing 0.5% FBS. Next, ECM digests were added to the media (0.1 mg/mL) with 0.5% FBS for 48 hrs. On the fourth day, culture media was replaced with media containing 10% ALAMAR BLUE (Invitrogen) and the cells were incubated for 14 hrs. Culture media were transferred into new 96-well plates and absorbance was measured at 570 nm and normalized to 600 nm.

LECM Growth Study

Lung cells were passaged by trypsinization after 7 days, by which time 70% to 80% confluency had been reached, seeded onto decellularized lung punches at an initial density of 2.5×10⁴ cells/mL, and cultured for 7 days. Cell growth was assessed by quantifying the DNA content of recellularized lung scaffolds with a Quant-iT PicoGreen dsDNA assay kit.

DNA Quantification of Seeded ECM Sheets and Hydrogels

DNA content of seeded ECM sheets and hydrogels was quantified using Quanti-iT PicoGreen dsDNA Assay kit (Invitrogen) according to the manufacturer's instructions. After 48 hrs of culture, samples were digested in 125 μg papain/mL overnight at 60° C. and mixed with PicoGreen reagent. Fluorescence emission was measured at 520 nm with excitation at 480 nm, and DNA was quantified using a standard curve.

Metabolic Activity

Kidney ECM sheets were glued to the bottom of 96-well plates using 2% fibrin. ECM and collagen I hydrogels were prepared in 96-well plates by adding 50 μL of hydrogel (neutralized and brought to the concentration of 6 mg/ml) at 4° C. The plates containing the hydrogels were incubated for 40 minutes at 37° C. until gelation was observed. KSCs and MSCs were grown under standard culture conditions, trypsinized, seeded into the ECM sheets or hydrogels at 2.5×10⁴ cells/mL, and cultured for 48 hr or 7 days. After a 48-hr incubation, the culture media was replaced with media containing 10% ALAMAR BLUE (Invitrogen). After 14-hr incubation, media were transferred into new 96-well plates and absorbance was measured at 570 nm and normalized to 600 nm.

Metabolic activity of lung cells during the growth study was measured using Alamar Blue reagent according to the manufacturer's instructions (Life Technologies). The reagent was added at various time points to the cells in culture and incubated for 9 to 12 hours before samples were collected and absorbance was measured at 570 nm with reference wavelength of 600 nm.

Mechanical Testing

Mechanical testing for LECMs was conducted with an Instron testing machine Model 5848 with a 10-N load cell. With the use of a custom-made mold, samples were cut into3-cm ×1-cm pieces from randomly selected transverse sections of the lower left lobe. This single orientation and the lower left lobe were selected and consistently maintained to minimize the effects of lung anisotropy on mechanical data. The 2 ends of the strips were secured with sandpaper to prevent slippage and mounted on the Instron, and a preload of 0.003 N was set. Samples were kept hydrated with 1× PBS at room temperature. Uniaxial stretch of 20% was applied at a rate of 1% strain/-second and frequencies of 0.25, 0.50, or 0.75 Hz (all samples were tested at the same grip-to-grip distance for consistency).

For cardiac ECMs, the hydrogel solutions were polymerized in custom-made molds to obtain disks (12 mm in diameter, 3 mm thick) that were transferred between two flat porous platens into a shear-strain controlled rheometer (ARESLS1, TA instrumentr, New Castle, Del.). A dynamic shear test was performed on a logarithmic frequency sweep (0.1-5 Hz) with shear strain amplitude of 0.1 rad. The complex shear modulus and loss tangent was calculated from the storage modulus and the loss modulus by standard equations.

Confocal Imaging

KSCs and MSCs grown under standard culture conditions were seeded into ECM sheets or hydrogels at 2.5×10⁴ cells/mL and cultured for 48 hrs or 7 days, at which times they were stained with Live/Dead Viability Kit (Invitrogen) or fixed with 3.7% formaldehyde and stained with rhodamine phalloidin (Invitrogen) and DAPI according to the manufacturer's instructions. Lung cells were stained with a Live/Dead kit (Invitrogen). Calcein-AM was used to indicate live cells (green), and ethidium homodimer-1 was used to indicate dead cells (red). Confocal imaging was performed using an Olympus Fluoview FV1000 Confocal Microscope.

Gelation Kinetics

Regional kidney ECM hydrogels and collagen I hydrogels were prepared as previously described. Gelation kinetics were determined spectrophotometrically. Briefly, gel solutions at 4° C. were transferred to a cold 96-well plate by adding 100 μL/well in triplicate. The SpectraMax spectrophotometer was pre-heated to 37° C., plate was loaded, and turbidity measured at 405 nm every 2 min for 1.5 hrs. Absorbance values were recorded for each well and averaged. Three separate tests were performed on two separate batches of kidney ECM hydrogels.

The gelation kinetics of cardiac ECMs were determined by transferring 50 μl of the appropriate gel mixture into a 96-well plate at 4° C. and transferring the plate into the spectrophotometer which was warmed up to 37° C., and the optical density was measured at 405 nm every 2 min for 60 min.

Chemotaxis (Transwell) Assay

KSCs were cultured for 24 hrs in 0.5% FBS, trypsinized, and seeded onto transwells with 8 μm pores. Region solubilized kidney ECM was added to the media at a concentration of 0.1 mg/mL. After 6 hrs, transwells were collected, attached cells removed from the top of the membrane using a Q-tip, and membranes were detached. DNA from cells attached to the bottoms of the detached membranes was quantified with CyQuant Direct Cell Proliferation Assay Kit according to the manufacturer's instructions. Fluorescence emission was measured at 535 nm with excitation at 480 nm, and DNA was quantified using a standard curve.

Statistical Analysis

One-way ANOVA test with Tukey's multiple comparison post hoc test and two-way ANOVA test with Bonferroni post hoc test were performed using Prism v6 (GraphPad, La Jolla, Calif.). A p<0.05 was considered statistically significant.

Human ESC Culture

The human ESCs (NIH code ES02) were obtained from ES Cell International. Briefly, hESCs were grown on a layer of mitomycin-treated mouse embryonic fibroblasts (Invitrogen) in hESC culture medium containing DMFM/F12 (Mediatech, Herndon, Va.) and supplemented with 20% knockout serum replacement, 10 mM nonessential amino acid, 200 mM glutamine, 1% penicillin/streptomycin, 0.2 μM β-mercaptoethariol (Sigma, St. Louis, Mo.), MEFconditioned medium, and 20 ng/ml bFGF (Invitrogen). Cells were incubated at 37° C. and 5% CO2. Medium was changed daily, and ESCs were split every 5 days using standard procedures.

Cardiac Differentiation of Human ESCs

Briefly, human ESCs were feeder-depleted by culture on a thin layer of Matrigel (BD Biosciences, Bedford, Mass.) in hESC culture medium for 24-48 h. EBs were formed by plating small aggregates of human ESCs in 2 ml basic medium (StemPro34, Invitrogen) containing 200 mM glutamine, 0.4 monothioglycerol, 5 μg/ml ascorbic acid (Sigma), and 0.5 ng/ml BMP4 (314-BP, R&D Systems). The factors were added in the following sequence: days 1-4: 10 ng/ml BMP4, bFGF (13256-029, Invitrogen), and 3 ng/ml activin A (338-AC/CF, R&D Systems); days 4-8: 10 ng/ml VEGF (293-VE, R&D Systems) and 150 ng/ml DKK1 (1096-DK/CF, R&D Systems); after day 8: 10 ng/ml VEGF, and 5 ng/ml bFGF. Cultures were maintained in a 5% CO₂/5% O₂/90% N₂ environment for 12 days of differentiation.

Gene Expression Analysis

RNA was extracted from culture using TRIzol reagent (Sigma) and standard isolation techniques. Concentration and purity were assessed with a NanoDrop 1000 spectrophotometer (Thermo Fisher Scientific, NH). Following conversion to cDNA using a High-Capacity cDNA Reverse Transcription Kit (Applied Biosystems, Calif.), quantitative reverse transcription polymerase chain reaction was conducted to analyze gene expression according to manufacturer's recommendations. Briefly, TagMan Gene Expression Assays (Applied Biosystems) were performed in 20-μL reaction volumes with TaqMan Fast Universal PCR Master Mix (Applied Biosystems) and the sample cDNA. Gene expression of TNNT2 relative to GAPDH, the endogenous control, was quantified via a 7500 Fast Real-Time PCR sequence detection system (Applied Biosystems) using the comparative CT methods. Data were expressed as a foldchange of the ratio in expression of TNNT2 and GAPDH.

Analysis of Contractile Function

EBs grown in hydrogels were observed under phase contrast microscope (Olympus IX81) at 37° C., and 200 frames of videos were taken at frame rate of 35.89 ms/frame. EB size was determined by pixel counting using ImageJ software (NIH) and analyzed with Microsoft Excel. The beating areas were defined as areas with the contraction displacement of >1%. Contraction amplitude was determined as the fractional change of the EB area.

While the disclosed subject matter is described herein in terms of certain exemplary embodiments, those skilled in the art will recognize that various modifications and improvements may be made to the disclosed subject matter without departing from the scope thereof. Moreover, although individual features of one embodiment of the disclosed subject matter may be discussed herein or shown in the drawings of the one embodiment and not in other embodiments, it should be apparent that individual features of one embodiment may be combined with one or more features of another embodiment or features from a plurality of embodiments.

In addition to the specific embodiments claimed below, the disclosed subject matter is also directed to other embodiments having any other possible combination of the dependent features claimed below and those disclosed above. As such, the particular features presented in the dependent claims and disclosed above can be combined with each other in other manners within the scope of the disclosed subject matter such that the disclosed subject matter should be recognized as also specifically directed to other embodiments having any other possible combinations. Thus, the foregoing description of specific embodiments of the disclosed subject matter has been presented for purposes of illustration and description. It is not intended to be exhaustive or to limit the disclosed subject matter to those embodiments disclosed.

It will be apparent to those skilled in the art that various modifications and variations can be made in the method and system of the disclosed subject matter without departing from the spirit or scope of the disclosed subject matter. Thus, it is intended that the disclosed subject matter include modifications and variations that are within the scope of the appended claims and their equivalents.

What is claimed is:

1. An engineered tissue comprising: tissue-derived decellularized extracellular matrix, wherein the tissue derived decellularized extracellular matrix is region specific.

2. The engineered tissue of claim 1, wherein the decellularized extracellular matrix is derived from a tissue selected from the group consisting of adrenal gland, amnion, bladder, blood vessel, bone, brain, breast, cartilage, chorion, connective tissue, esophagus, eye, fat, heart, kidney, large intestine, larynx, ligament, liver, lung, lymph node, microvasculature, muscle, omentum, ovary, pancreas, placenta membrane, prostate, skin, small intestine, smooth muscle, spinal cord, spleen, stomach, tendon, testis, thymus, umbilical cord, uterus, or Wharton's Jelly.

3. The engineered tissue of claim 1, wherein the engineered tissue is a scaffold, sponge, solution, hydrogel, fiber, bio-ink, patch or interposition graft.

4. The engineered tissue of claim 1, wherein the regionally specific tissue derived decellularized extracellular matrix is metabolically active.

5. The engineered tissue of claim 1, further comprising tissue stem cells seeded on the tissue-derived decellularized extracellular matrix.

6. The engineered tissue of claim 1, further comprising a solution and at least one reagent adapted to reconstitute the solution into a hydrogel, wherein the engineered tissue is in the solution.

7. A method for treating damaged tissue comprising implanting the engineered tissue of claim 1.

8. A method of treating a subject with regenerating tissue comprising implanting the engineered tissue of claim 1.

9. A method comprising extracting a regionally specific portion of a tissue derived from a tissue selected from the group consisting of adrenal gland, amnion, bladder, blood vessel, bone, brain, breast, cartilage, chorion, connective tissue, esophagus, eye, fat, heart, kidney, large intestine, larynx, ligament, liver, lung, lymph node, microvasculature, muscle, omentum, ovary, pancreas, placenta membrane, prostate, skin, small intestine, smooth muscle, spinal cord, spleen, stomach, tendon, testis, thymus, umbilical cord, uterus, or Wharton's Jelly; decellularizing the portion of the tissue to yield an extracellular matrix; powdering the extracellular matrix to yield a powder; digesting the powder to yield a digest; processing the digest to a tissue-derived decellularized extracellular matrix.

10. The method of claim 9, wherein the processing step includes reconstituting the digest to form a hydrogel.

11. The method of claim 9, wherein the processing step includes centrifuging, vortexing and lyophilizing the digest to form a sponge.

12. The method of claim 9, wherein the processing step includes electrospinning the digest to form fibers.

13. The method of claim 9, wherein the processing step includes formulating the digest into a bio-ink and using the bio-ink to print the tissue-derived decellularized extracellular matrix.

14. The method of claim 9, further including culturing stem cells on the tissue-derived decellularized matrix.

15. The method of claim 14, wherein the stem cells are mesenchymal stem cells.

16. A method for generating a tissue ex vivo, the method comprising:
generating a biomaterial including tissue-derived decellularized extracellular matrix, wherein the tissue-derived decellularized extracellular matrix is region specific; and seeding the biomaterial with at least one type of stem cell providing conditions to allow the stem cells to generate on the biomaterial.

17. The method of claim 16, wherein the tissue-derived decellularized extracellular matrix is selected from the group consisting of adrenal gland, amnion, bladder, blood vessel, bone, brain, breast, cartilage, chorion, connective tissue, esophagus, eye, fat, heart, kidney, large intestine, larynx, ligament, liver, lung, lymph node, microvasculature, muscle, omentum, ovary, pancreas, placenta membrane, prostate, skin, small intestine, smooth muscle, spinal cord, spleen, stomach, tendon, testis, thymus, umbilical cord, uterus, stem cells or Wharton's Jelly.

18. The method of claim 16, wherein the stem cells are mesenchymal stem cells.
